(19) United States
(12) Patent Application Publication
(10) Pub. No.: US 2023/0174674 A1
(43) Pub. Date: Jun. 8, 2023

(54) PLASMA KALLIKREIN INHIBITORS AND USES THEREOF FOR TREATING ACUTE RESPIRATORY DISTRESS SYNDROME

(71) Applicant: Takeda Pharmaceutical Company Limited, Osaka-shi, Osaka (JP)

(72) Inventors: Daniel J. Sexton, Melrose, MA (US); Neil Inhaber, Westborough, MA (US); Giorgio Giannattasio, Cham (CH); Donatello Crocetta, Cham (CH)

(73) Assignee: Takeda Pharmaceutical Company Limited, Osaka-shi, Osaka (JP)

(21) Appl. No.: 17/916,621

(22) PCT Filed: Apr. 2, 2021
(86) PCT No.: PCT/US2021/025495

§ 371 Date: Oct. 3, 2022

Related U.S. Application Data
(60) Provisional application No. 63/086,921, filed on Oct. 2, 2020.
(60) Provisional application No. 63/037,838, filed on Jun. 11, 2020.
(60) Provisional application No. 63/005,251, filed on Apr. 4, 2020.

(51) Int. Cl. C07K 16/40; A61K 39/395; A61P 31/14; A61P 11/00; A61K 38/08; C07K 16/28; A61K 31/513; A61K 31/427; A61K 38/21; A61K 31/404; A61K 31/675; A61K 31/4706

(52) U.S. Cl. CPC C07K 16/40; A61K 31/404; A61K 31/427; A61K 31/513; A61K 31/675; A61K 31/4706; A61K 38/08; A61K 38/215; A61K 39/3955; A61P 11/00; A61P 31/14; C07K 16/2866; A61K 2039/505; C07K 2317/565

(57) ABSTRACT

Provided herein are methods for treating acute respiratory distress syndrome (ARDS), such as ARDS associated with respiratory virus infection, involving administering an inhibitor of the contact activation pathway. Also provided herein are methods for treating pneumonia and methods for reducing and/or preventing thrombosis associated with extracorporeal membrane oxygenation (ECMO) in a subject having acute respiratory distress syndrome (ARDS), involving administering an inhibitor of the contact activation pathway.

### RELATED APPLICATIONS

This application claims the benefit under 35 U.S.C. § 119(e) of U.S. Provisional Application No. 63/005,251, filed Apr. 4, 2020; U.S. Provisional Application No. 63/037,838, filed Jun. 11, 2020; and U.S. Provisional Application No. 63/086,921, filed Oct. 2, 2020, the contents of each of which are incorporated by reference herein in its entirety.

### BACKGROUND

Acute respiratory distress syndrome (ARDS) is a life-threatening condition that is induced by an inflammatory reaction following lung trauma. Pulmonary inflammation leads to immune responses that weaken blood vessels and cause fluid leakage into alveoli, which decreases oxygen supply, resulting in respiratory distress. In general, ARDS onset may be attributed to a respiratory viral infection, a blood infection, inhalation of toxic substances, an injury to the chest or head, or an overdose of sedatives or tricyclic antidepressants.

### SUMMARY

Aspects of the present disclosure provide methods for treating acute respiratory distress syndrome (ARDS), methods of treating thrombosis associated with extracorporeal membrane oxygenation (ECMO) in a patient having ARDS, and methods of treating pneumonia using inhibitors of the contact activation pathway, for example, antibodies having the same complementarity determining regions (CDRs) as lanadelumab (a.k.a. DX-2930, SHP463).

In some aspects, the present disclosure provides a method for treating acute respiratory distress syndrome (ARDS) comprising administering to a subject in need thereof an inhibitor of the contact activation pathway. In some embodiments, the subject is a human subject. In some embodiments, the human subject has or is suspected of having a viral infection. In some embodiments, the viral infection is a respiratory viral infection.

In some embodiments, the ARDS is associated with a respiratory viral infection, a blood infection, pancreatitis, inhalation of toxic substances, an injury to the chest or head, or an overdose of sedatives or tricyclic antidepressants. In some embodiments the respiratory viral infection is a coronavirus infection. In some embodiments, the coronavirus infection is infection with severe acute respiratory coronavirus 2 (SARS-CoV-2, COVID-19).

In some embodiments the subject has one or more symptom of a respiratory viral infection. In some embodiments, the subject has pneumonia associated with a respiratory viral infection. In some embodiments, the one or more symptom of the respiratory viral infection is fever, cough, fatigue, sputum production, loss of smell, loss of taste, shortness of breath, muscle or joint pain, sore throat, headache, chills, nausea or vomiting, nasal congestion, diarrhea, haemoptysis, and/or conjunctival congestion. In some embodiments, the ARDS is associated with inhalation of toxic substances.

In some embodiments, the inhibitor of the contact activation pathway is a plasma kallikrein (pKal) inhibitor. In some embodiments, the pKal inhibitor is an anti-pKal antibody. In some embodiments, the anti-pKal antibody comprises heavy chain complementarity determining regions set forth by SEQ ID NOs: 5-7 and light chain variable complementarity determining regions set forth by SEQ ID NOs: 8-10. In some embodiments, the anti-pKal antibody is a full-length antibody or an antigen-binding fragment thereof. In some embodiments, the anti-pKal antibody comprises a heavy chain variable region set forth by SEQ ID NO: 3 and/or a light chain variable region set forth by SEQ ID NO: 4. In some embodiments, the anti-pKal antibody comprises a heavy chain set forth by SEQ ID NO: 1 and a light chain set forth by SEQ ID NO: 2. In some embodiments, the anti-pKal antibody comprises a heavy chain set forth by SEQ ID NO: 11 and a light chain set forth by SEQ ID NO: 12.

In some embodiments, the anti-pKal antibody is formulated in a pharmaceutical composition comprising a pharmaceutical composition comprising a pharmaceutically acceptable carrier. In some embodiments the pharmaceutical composition comprises sodium phosphate, citric acid, histidine, sodium chloride, and polysorbate 80. In some embodiments, the sodium phosphate is at a concentration of about 30 mM, the citric acid is at a concentration of about 19 mM, the histidine is at a concentration of about 50 mM, the sodium chloride is at a concentration of about 90 mM, and the polysorbate 80 is at about 0.01%.

In some embodiments, the anti-pKal antibody is administered in one or more doses. In some embodiments, each of the one or more doses comprises about 100 mg - about 400 mg of the antibody. In some embodiments, each of the one or more doses comprises about 300 mg of the antibody. In some embodiments, the antibody is administered to the subject every two weeks. In some embodiments, the antibody is administered to the subject in one dose. In some embodiments, the antibody is administered to the subject every three days. In some embodiments, the antibody is administered subcutaneously. In some embodiments, the antibody is administered intravenously, such as by intravenous infusion.

In some embodiments, the method further comprises administering one or more additional therapeutic agent to the subject. In some embodiments, the subject has been administered one or more additional therapeutic agent prior to administering the inhibitor of the contact activation pathway. In some embodiments, the one or more additional therapeutic agent is an immunomodulatory agent, an antiviral agent, an anti-malarial agent, and/or an additional inhibitor of the contact activation pathway. In some embodiments, the immunomodulatory agent is an inhibitor of IL-6R. In some embodiments, the inhibitor of IL-6R is tocilizumab or sarilumab. In some embodiments, the antiviral agent is lopinavir, ritonavir, interferon beta, umfenovir, remdesivir, or a combination thereof. In some embodiments, the anti-malarial agent is chloroquine. In some embodiments, the additional inhibitor of the contact activation pathway is a C1-inhibitor, a pKal inhibitor, or a bradykinin receptor antagonist. In some embodiments, the bradykinin receptor antagonist is icatibant.

In some aspects, the present disclosure provides a method for preventing thrombosis associated with extracorporeal membrane oxygenation (ECMO) in a subject having acute respiratory distress syndrome (ARDS), the method comprising administering to a subject in need thereof an inhibitor of the contact activation pathway.

In some embodiments, the ARDS is associated with a respiratory viral infection, a blood infection, pancreatitis, inhalation of toxic substances, an injury to the chest or head, or an overdose of sedatives or tricyclic antidepressants. In some embodiments the respiratory viral infection is a coronavirus infection. In some embodiments, the coronavirus infection is infection with severe acute respiratory coronavirus 2 (SARS-CoV-2, COVID-19).

In some embodiments the subject has one or more symptom of a respiratory viral infection. In some embodiments, the subject has pneumonia associated with a respiratory viral infection. In some embodiments, the one or more symptom of the respiratory viral infection is fever, cough, fatigue, sputum production, loss of smell, loss of taste, shortness of breath, muscle or joint pain, sore throat, headache, chills, nausea or vomiting, nasal congestion, diarrhea, haemoptysis, and/or conjunctival congestion. In some embodiments, the ARDS is associated with inhalation of toxic substances.

In some embodiments, the inhibitor of the contact activation pathway is a plasma kallikrein (pKal) inhibitor. In some embodiments, the pKal inhibitor is an anti-pKal antibody. In some embodiments, the anti-pKal antibody comprises heavy chain complementarity determining regions set forth by SEQ ID NOs: 5-7 and light chain variable complementarity determining regions set forth by SEQ ID NOs: 8-10. In some embodiments, the anti-pKal antibody is a full-length antibody or an antigen-binding fragment thereof. In some embodiments, the anti-pKal antibody comprises a heavy chain variable region set forth by SEQ ID NO: 3 and/or a light chain variable region set forth by SEQ ID NO: 4. In some embodiments, the anti-pKal antibody comprises a heavy chain set forth by SEQ ID NO: 1 and a light chain set forth by SEQ ID NO: 2. In some embodiments, the anti-pKal antibody comprises a heavy chain set forth by SEQ ID NO: 11 and a light chain set forth by SEQ ID NO: 12.

In some embodiments, the anti-pKal antibody is formulated in a pharmaceutical composition comprising a pharmaceutical composition comprising a pharmaceutically acceptable carrier. In some embodiments the pharmaceutical composition comprises sodium phosphate, citric acid, histidine, sodium chloride, and polysorbate 80. In some embodiments, the sodium phosphate is at a concentration of about 30 mM, the citric acid is at a concentration of about 19 mM, the histidine is at a concentration of about 50 mM, the sodium chloride is at a concentration of about 90 mM, and the polysorbate 80 is at about 0.01%.

In some embodiments, the anti-pKal antibody is administered in one or more doses. In some embodiments, each of the one or more doses comprises about 100 mg - about 400 mg of the antibody. In some embodiments, each of the one or more doses comprises about 300 mg of the antibody. In some embodiments, the antibody is administered to the subject every two weeks. In some embodiments, the antibody is administered to the subject in one dose. In some embodiments, the antibody is to the subject every three days. In some embodiments, the antibody is administered subcutaneously. In some embodiments, the antibody is administered intravenously, optionally by intravenous infusion.

In some embodiments, the method further comprises administering one or more additional therapeutic agent to the subject. In some embodiments, the subject has been administered one or more additional therapeutic agent prior to administering the inhibitor of the contact activation pathway.

In some embodiments, the one or more additional therapeutic agent is an immunomodulatory agent, an antiviral agent, an anti-malarial agent, and/or an additional inhibitor of the contact activation pathway. In some embodiments, the immunomodulatory agent is an inhibitor of IL-6R. In some embodiments, the inhibitor of IL-6R is tocilizumab or sarilumab. In some embodiments, the antiviral agent is lopinavir, ritonavir, interferon beta, umfenovir, remdesivir, or a combination thereof. In some embodiments, the anti-malarial agent is chloroquine. In some embodiments, the additional inhibitor of the contact activation pathway is a C1-inhibitor, a pKal inhibitor, or a bradykinin receptor antagonist. In some embodiments, the bradykinin receptor antagonist is icatibant.

In some aspects, the present disclosure provides a method for treating pneumonia, the method comprising administering to a subject in need thereof an inhibitor of the contact activation pathway. In some embodiments, the subject is a human subject. In some embodiments, the human subject has or is suspected of having a viral infection. In some embodiments, the viral infection is a respiratory viral infection. In some embodiments the respiratory viral infection is a coronavirus infection. In some embodiments, the pneumonia is associated with severe acute respiratory coronavirus 2 (SARS-CoV-2, COVID-19).

In some embodiments the subject has one or more symptom of a respiratory viral infection. In some embodiments, the one or more symptom of the respiratory viral infection is fever, cough, fatigue, sputum production, loss of smell, loss of taste, shortness of breath, muscle or joint pain, sore throat, headache, chills, nausea or vomiting, nasal congestion, diarrhea, haemoptysis, and/or conjunctival congestion.

In some embodiments, the inhibitor of the contact activation pathway is a plasma kallikrein (pKal) inhibitor. In some embodiments, the pKal inhibitor is an anti-pKal antibody. In some embodiments, the anti-pKal antibody comprises heavy chain complementarity determining regions set forth by SEQ ID NOs: 5-7 and light chain variable complementarity determining regions set forth by SEQ ID NOs: 8-10. In some embodiments, the anti-pKal antibody is a full-length antibody or an antigen-binding fragment thereof. In some embodiments, the anti-pKal antibody comprises a heavy chain variable region set forth by SEQ ID NO: 3 and/or a light chain variable region set forth by SEQ ID NO: 4. In some embodiments, the anti-pKal antibody comprises a heavy chain set forth by SEQ ID NO: 1 and a light chain set forth by SEQ ID NO: 2. In some embodiments, the anti-pKal antibody comprises a heavy chain set forth by SEQ ID NO: 11 and a light chain set forth by SEQ ID NO: 12.

In some embodiments, the anti-pKal antibody is formulated in a pharmaceutical composition comprising a pharmaceutical composition comprising a pharmaceutically acceptable carrier. In some embodiments the pharmaceutical composition comprises sodium phosphate, citric acid, histidine, sodium chloride, and polysorbate 80. In some embodiments, the sodium phosphate is at a concentration of about 30 mM, the citric acid is at a concentration of about 19 mM, the histidine is at a concentration of about 50 mM, the sodium chloride is at a concentration of about 90 mM, and the polysorbate 80 is at about 0.01%.

In some embodiments, the anti-pKal antibody is administered in one or more doses. In some embodiments, each of the one or more doses comprises about 100 mg - about 400 mg of the antibody. In some embodiments, each of the one or more doses comprises about 300 mg of the antibody. In some embodiments, the antibody is administered to the subject in one dose. In some embodiments, the antibody is administered to the subject every 3 days. In some embodiments, the antibody is administered intravenously, optionally by intravenous infusion.

In some embodiments, the method further comprises administering one or more additional therapeutic agent to the subject. In some embodiments, the subject has been administered one or more additional therapeutic agent prior to administering the inhibitor of the contact activation pathway.

In some embodiments, the one or more additional therapeutic agent is an antiviral agent and/or an anti-malarial agent. In some embodiments, the antiviral agent is lopinavir, ritonavir, interferon beta, umfenovir, remdesivir, or a combination thereof. In some embodiments, the anti-malarial agent is chloroquine.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

FIG. 1 is a schematic of the contact activation pathway, including inhibition of Factor XIIa and plasma kallikrein with intravenous C1-esterase inhibitor (C1-INH IV), and inhibition of plasma kallikrein with subcutaneous (“SC”) lanadelumab (DX-2930, SHP463) to inhibit intrinsic coagulation. HMWK is high molecular weight kininogen, B1R GPCR is bradykinin 1 receptor G-protein coupled receptor, BK is bradykinin, B2R GPCR is bradykinin 2 receptor G-protein coupled receptor, IL-1β is interleukin 1 beta, IL-6 is interleukin 6, IL-8 is interleukin 8, EGF is extracellular growth factor, CPN is carboxypeptidase-N, NF-kB is nuclear factor kappa-light-chain-enhancer of activated B cells, KKS is kallikrein-kinin system, and HKa is activated high molecular weight kininogen.

### DETAILED DESCRIPTION

### Definitions

For convenience, before further description of the present invention, certain terms employed in the specification, examples and appended claims are defined here. Other terms are defined as they appear in the specification.

The singular forms “a,” “an,” and “the” include plural references unless the context clearly dictates otherwise.

As used herein, the term “about” refers to a particular value +/- 5%. For example, an antibody at about 300 mg includes any amount of the antibody between 285 mg - 315 mg.

The term “antibody” refers to an immunoglobulin molecule capable of specific binding to a target, such as a carbohydrate, polynucleotide, lipid, polypeptide, etc., through at least one antigen recognition site located in the variable region of the immunoglobulin molecule. An antibody may include at least one heavy (H) chain that comprises a heavy chain immunoglobulin variable domain (V_{H}), at least one light chain that comprises a light chain immunoglobulin variable domain (V_{L}), or both. For example, an antibody can include a heavy (H) chain variable region (abbreviated herein as V_{H} or VH) and a light (L) chain variable region (abbreviated herein as V_{L} or VL). In another example, an antibody includes two heavy (H) chain variable regions and two light (L) chain variable regions.

As used herein, the term “antibody” encompasses not only intact (i.e., full-length) polyclonal or monoclonal antibodies, but also antigen-binding fragments thereof (such as Fab, Fab′, F(ab′)₂, Fv), single chain (scFv), domain antibody (dAb) fragments (de Wildt et. al., Euro. J. Immunol. (1996) 26(3): 629-639), any mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Antibodies may be from any source, but primate (human and non-human primate) and primatized are preferred.

The V_{H} and/or V_{L} regions may include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that includes immunoglobulin variable domain sequence can associate with another immunoglobulin variable domain sequence to form an antigen binding site, e.g., a structure that preferentially interacts with plasma kallikrein.

The V_{H} and V_{L} regions can be further subdivided into regions of hypervariability, termed “complementarity determining regions” (“CDRs”), interspersed with regions that are more conserved, termed “framework regions” (“FRs”). The extent of the framework region and CDRs have been defined (see, Kabat, E.A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). Kabat definitions are used herein. Each VH and VL is typically composed of three CDRs and four FRs, arranged from amino-terminus to carboxy-terminus in the following order: FR1, CDR1, FR2, CDR2, FR3, CDR3, FR4.

In addition to the V_{H} or V_{L} regions, the heavy chain or light chain of the antibody can further include all or part of a heavy or light chain constant region. In one embodiment, the antibody is a tetramer of two heavy immunoglobulin chains and two light immunoglobulin chains, wherein the heavy and light immunoglobulin chains are inter-connected by, e.g., disulfide bonds. In IgGs, the heavy chain constant region includes three immunoglobulin domains, CH1, CH2 and CH3. The light chain constant region includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The light chains of the immunoglobulin may be of type kappa or lambda. In one embodiment, the antibody is glycosylated. An antibody can be functional for antibody-dependent cytotoxicity and/or complement-mediated cytotoxicity.

One or more regions of an antibody can be human or effectively human. For example, one or more of the variable regions can be human or effectively human. For example, one or more of the CDRs can be human, e.g., HC CDR1, HC CDR2, HC CDR3, LC CDR1, LC CDR2, and/or LC CDR3. Each of the light chain (LC) and/or heavy chain (HC) CDRs can be human. HC CDR3 can be human. One or more of the framework regions can be human, e.g., FR1, FR2, FR3, and/or FR4 of the HC and/or LC. For example, the Fc region can be human. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. In one embodiment, the framework (FR) residues of a selected Fab can be converted to the amino-acid type of the corresponding residue in the most similar primate germline gene, especially the human germline gene. One or more of the constant regions can be human or effectively human. For example, at least 70, 75, 80, 85, 90, 92, 95, 98, or 100% of an immunoglobulin variable domain, the constant region, the constant domains (CH1, CH2, CH3, and/or CL1), or the entire antibody can be human or effectively human.

An antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the many immunoglobulin variable region genes. Full-length immunoglobulin “light chains” (about 25 KDa or about 214 amino acids) are encoded by a variable region gene at the NH2-terminus (about 110 amino acids) and a kappa or lambda constant region gene at the COOH-terminus. Full-length immunoglobulin “heavy chains” (about 50 KDa or about 446 amino acids), are similarly encoded by a variable region gene (about 116 amino acids) and one of the other aforementioned constant region genes, e.g., gamma (encoding about 330 amino acids). The length of human HC varies considerably because HC CDR3 varies from about 3 amino-acid residues to over 35 amino-acid residues.

The term “antigen-binding fragment” of a full length antibody refers to one or more fragments of a full-length antibody that retain the ability to specifically bind to a target of interest. Examples of binding fragments encompassed within the term “antigen-binding fragment” of a full length antibody and that retain functionality include (i) a Fab fragment, a monovalent fragment consisting of the V_{L}, V_{H}, C_{L} and CH1 domains; (ii) a F(ab′)2 fragment, a bivalent fragment including two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., U.S. Pat. Nos. 5,260,203, 4,946,778, and 4,881,175; Bird et al. (1988) Science 242:423-426; and Huston et al. (1988) Proc. Natl. Acad. Sci. USA 85:5879-5883. Antibody fragments can be obtained using any appropriate technique including conventional techniques known to those with skill in the art.

The term “monospecific antibody” refers to an antibody that displays a single binding specificity and affinity for a particular target, e.g., epitope. This term includes a “monoclonal antibody” or “monoclonal antibody composition,” which as used herein refers to a preparation of antibodies or fragments thereof of single molecular composition, irrespective of how the antibody was generated. Antibodies are “germlined” by reverting one or more non-germline amino acids in framework regions to corresponding germline amino acids of the antibody, so long as binding properties are substantially retained.

The inhibition constant (Ki) provides a measure of inhibitor potency; it is the concentration of inhibitor required to reduce enzyme activity by half and is not dependent on enzyme or substrate concentrations. The apparent Kᵢ (K_{i,app}) is obtained at different substrate concentrations by measuring the inhibitory effect of different concentrations of inhibitor (e.g., inhibitory binding protein) on the extent of the reaction (e.g., enzyme activity); fitting the change in pseudo-first order rate constant as a function of inhibitor concentration to the Morrison equation (Equation 1) yields an estimate of the apparent Ki value. The Ki is obtained from the y-intercept extracted from a linear regression analysis of a plot of K_{i,app} versus substrate concentration.

$$v = {v}_{o} - {v}_{o} \left(\frac{\left({K}_{i , a p p} + I + E\right) - \sqrt{{\left({K}_{i , a p p} + I + E\right)}^{2} - 4 ⋅ I ⋅ E}}{2 ⋅ E}\right)$$

Where v = measured velocity; v0 = velocity in the absence of inhibitor; K_{i,app} = apparent inhibition constant; I = total inhibitor concentration; and E = total enzyme concentration.

As used herein, “binding affinity” refers to the apparent association constant or K_{A}. The K_{A} is the reciprocal of the dissociation constant (K_{D}). A binding antibody may, for example, have a binding affinity of at least 10⁵, 10⁶, 10⁷, 10⁸, 10⁹, 10¹⁰ and 10¹¹ M⁻¹ for a particular target molecule, e.g., plasma kallikrein. Higher affinity binding of a binding antibody to a first target relative to a second target can be indicated by a higher K_{A} (or a smaller numerical value K_{D}) for binding the first target than the K_{A} (or numerical value K_{D}) for binding the second target. In such cases, the binding antibody has specificity for the first target (e.g., a protein in a first conformation or mimic thereof) relative to the second target (e.g., the same protein in a second conformation or mimic thereof; or a second protein). Differences in binding affinity (e.g., for specificity or other comparisons) can be at least 1.5, 2, 3, 4, 5, 10, 15, 20, 30, 40, 50, 70, 80, 90, 100, 500, 1000, 10,000, or 10⁵-fold.

Binding affinity can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH 7.4, 150 mM NaCl, 0.005% (v/v) Surfactant P20). These techniques can be used to measure the concentration of bound and free binding protein as a function of binding protein (or target) concentration. The concentration of bound binding protein ([Bound]) is related to the concentration of free binding protein ([Free]) and the concentration of binding sites for the binding protein on the target where (N) is the number of binding sites per target molecule by the following equation: [Bound] = N • [Free]/((1/KA) + [Free]).

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2 fold higher, to obtain a qualitative measurement of affinity, or to obtain an inference of affinity, e.g., by activity in a functional assay, e.g., an in vitro or in vivo assay.

The term “binding antibody” (or “binding protein” used interchangeably herein) refers to an antibody that can interact with a target molecule. The term “target molecule” is used interchangeably with “ligand.” A “plasma kallikrein binding antibody” refers to an antibody that can interact with (e.g., bind) plasma kallikrein, and includes, in particular, antibodies that preferentially or specifically interact with and/or inhibit plasma kallikrein. An antibody inhibits plasma kallikrein if it causes a decrease in the activity of plasma kallikrein as compared to the activity of plasma kallikrein in the absence of the antibody and under the same conditions.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

It is possible for one or more framework and/or CDR amino acid residues of a binding protein to include one or more mutations (for example, substitutions (e.g., conservative substitutions or substitutions of non-essential amino acids), insertions, or deletions) relative to a binding protein described herein. A plasma kallikrein binding protein may have mutations (e.g., substitutions (e.g., conservative substitutions or substitutions of non-essential amino acids), insertions, or deletions) (e.g., at least one, two, three, or four, and/or less than 15, 12, 10, 9, 8, 7, 6, 5, 4, 3, or 2 mutations) relative to a binding protein described herein, e.g., mutations which do not have a substantial effect on protein function. The mutations can be present in framework regions, CDRs, and/or constant regions. In some embodiments, the mutations are present in a framework region. In some embodiments, the mutations are present in a CDR. In some embodiments, the mutations are present in a constant region. Whether or not a particular substitution will be tolerated, i.e., will not adversely affect biological properties, such as binding activity, can be predicted, e.g., by evaluating whether the mutation is conservative or by the method of Bowie, et al. (1990) Science 247:1306-1310.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

An “epitope” refers to the site on a target compound that is bound by a binding protein (e.g., an antibody such as a Fab or full-length antibody). In the case where the target compound is a protein, the site can be entirely composed of amino acid components, entirely composed of chemical modifications of amino acids of the protein (e.g., glycosyl moieties), or composed of combinations thereof. Overlapping epitopes include at least one common amino acid residue, glycosyl group, phosphate group, sulfate group, or other molecular feature.

A “humanized” immunoglobulin variable region is an immunoglobulin variable region that is modified to include a sufficient number of human framework amino acid positions such that the immunoglobulin variable region does not elicit an immunogenic response in a normal human. Descriptions of “humanized” immunoglobulins include, for example, U.S. 6,407,213 and U.S. 5,693,762.

An “isolated” antibody refers to an antibody that is removed from at least 90% of at least one component of a natural sample from which the isolated antibody can be obtained. Antibodies can be “of at least” a certain degree of purity if the species or population of species of interest is at least 5, 10, 25, 50, 75, 80, 90, 92, 95, 98, or 99% pure on a weight-weight basis.

The methods described herein involve administering multiple doses of an antibody to a human subject in need thereof. The terms “patient,” “subject,” or “host” may be used interchangeably. In some embodiments, the subject is a pediatric subject (e.g., an infant, child, or adolescent subject). In some embodiments, the human subject is an adolescent less than 18 years old. In some embodiments, the human subject is an adolescent between the ages of 12 and 18 years old. In some embodiments, the subject is between the ages of 40 and less than 65 years old. In some embodiments, the subject is a geriatric subject (e.g., a subject over 65 years old).

In some embodiments, the human subject is defined by gender. For example, in some embodiments, the subject is female. In some embodiments, the subject is male.

The terms “prekallikrein” and “preplasma kallikrein” are used interchangeably herein and refer to the zymogen form of active plasma kallikrein, which is also known as prekallikrein.

As used herein, the term “substantially identical” (or “substantially homologous”) is used herein to refer to a first amino acid or nucleic acid sequence that contains a sufficient number of identical or equivalent (e.g., with a similar side chain, for example, conserved amino acid substitutions) amino acid residues or nucleotides to a second amino acid or nucleic acid sequence such that the first and second amino acid or nucleic acid sequences have (or encode proteins having) similar activities, e.g., a binding activity, a binding preference, or a biological activity. In the case of antibodies, the second antibody has the same specificity and has at least 50%, at least 25%, or at least 10% of the affinity relative to the same antigen.

Statistical significance can be determined by any art known method. Exemplary statistical tests include: the Students T-test, Mann Whitney U non-parametric test, and Wilcoxon non-parametric statistical test. Some statistically significant relationships have a P value of less than 0.05 or 0.02. Particular binding proteins may show a difference, e.g., in specificity or binding that are statistically significant (e.g., P value < 0.05 or 0.02). The terms “induce,” “inhibit,” “potentiate,” “elevate,” “increase,” “decrease” or the like, e.g., which denote distinguishable qualitative or quantitative differences between two states, may refer to a difference, e.g., a statistically significant difference, between the two states.

A “therapeutically effective dosage” preferably modulates a measurable parameter, e.g., one or more symptom or measure of ARDS, by a statistically significant degree or at least about 20%, more preferably by at least about 40%, even more preferably by at least about 60%, and still more preferably by at least about 80% relative to untreated subjects. The ability of a compound to modulate a measurable parameter, e.g., a disease-associated parameter, can be evaluated in an animal model system predictive of efficacy in human disorders and conditions. Alternatively, this property of a composition can be evaluated by examining the ability of the compound to modulate a parameter in vitro.

The term “treating” as used herein refers to the application or administration of a composition including one or more active agents to a subject, who has acute respiratory distress syndrome (ARDS), a symptom of ARDS, is suspected of having ARDS, or a predisposition toward or risk of having ARDS, with the purpose to cure, heal, alleviate, relieve, alter, remedy, ameliorate, improve, or affect the disease, the symptoms of the disease, or the predisposition toward the disease. “Prophylactic treatment,” also known as “preventive treatment,” refers to a treatment that aims at protecting a person from, or reducing the risk for, ARDS. In some embodiments, the treatment methods described herein aim at preventing or delaying ARDS or a symptom thereof. In some embodiments, the treatment methods described herein aim at preventing or delaying respiratory failure. In some embodiments, the treatment methods described herein aim at preventing death associated with ARDS.

The term “preventing” a disease in a subject refers to subjecting the subject to a pharmaceutical treatment, e.g., the administration of a drug, such that at least one symptom of ARDS is prevented, that is, administered prior to clinical manifestation of the unwanted condition so that it protects the host against developing the unwanted condition.

A “prophylactically effective amount” refers to an amount effective, at dosages and for periods of time necessary, to achieve the desired prophylactic result. Typically, because a prophylactic dose is used in subjects prior to or at an earlier stage of disease, the prophylactically effective amount will be less than the therapeutically effective amount.

A “pneumonia associated with a respiratory viral infection” is a pneumonia that either occurs prior to and causes a respiratory viral infection or a pneumonia that occurs after and is caused by a respiratory viral infection. A pneumonia that occurs prior to a respiratory viral infection may compromise the integrity of the respiratory system (e.g., bronchi, bronchioles, alveoli, trachea, etc.), allowing a respiratory virus to infect one or both lungs. A pneumonia that occurs after a respiratory viral infection may affect lung tissue that has been compromised by the respiratory viral infection.

### Inhibitors of the Contact Activation Pathway

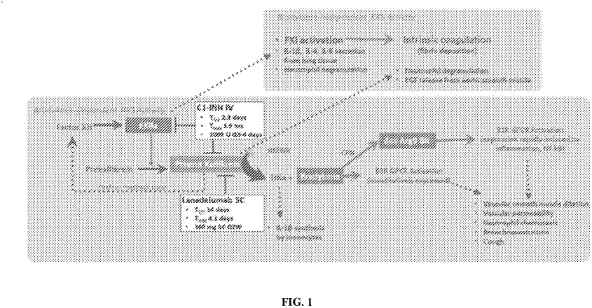

The contact activation system involves a series of plasma proteins and proteases including Factor XII, plasma kallikrein, and high molecular weight kininogen, activation of which promotes inflammation and blood coagulation (Simao et al. Front. Med. (2017)). Plasma kallikrein (PKal) is largely responsible for the generation of bradykinin in the vasculature and circulates as an inactive zymogen called prekallikrein that is mostly bound to its substrate, high molecular weight kininogen (HMWK). In response to a stimulus, FXII is activated to FXIIa, which cleaves prekallikrein to form active plasma kallikrein (FIG. 1). Approximately 75-90% of circulating prekallikrein is bound to HMWK through a non-active site interaction with domain 6 of HMWK. Free and HMWK-bound active pKal generate cleaved HMWK and the proinflammatory nonapeptide bradykinin.

Excess contact system activation leads to elevated plasma kallikrein activity and bradykinin levels that can induce excess vascular smooth muscle dilation, vascular permeability, neutrophil chemotaxis, bronchoconstriction, and cough (Kolte, et al., PF-04886847 (an inhibitor of plasma kallikrein) attenuates inflammatory mediators and activation of blood coagulation in rat model of lipopolysaccharide (LPS)-induced sepsis; Cardiovasc Hematol Agents Med Chem, 2012, 10(2): 154-166).

Provided herein are methods of treating acute respiratory distress syndrome involving administering an inhibitor of the contact activation pathway. In some embodiments, the inhibitor of the contact activation pathway is a plasma kallikrein inhibitor. In some embodiments, the inhibitor of the contact activation pathway is an antibody. In some embodiments, the inhibitor of the contact activation pathway is a plasma kallikrein antibody (also referred to as an anti-plasma kallikrein antibody).

Antibodies that inhibit the contact activation pathway (e.g., inhibit one or more components involved in the contact activation pathway) for use in the methods described herein can be full-length (e.g., an IgG (including an IgG1, IgG2, IgG3, IgG4), IgM, IgA (including, IgA1, IgA2), IgD, and IgE) or can include only an antigen-binding fragment (e.g., a Fab, F(ab′)₂ or scFv fragment. The binding antibody can include two heavy chain immunoglobulins and two light chain immunoglobulins, or can be a single chain antibody. Antibodies that inhibit the contact activation pathway can be recombinant proteins such as humanized, CDR grafted, chimeric, deimmunized, or in vitro generated antibodies, and may optionally include constant regions derived from human germline immunoglobulin sequences. In some embodiments, the antibody that inhibits the contact activation pathway is an antibody that binds and inhibits plasma kallikrein. In some embodiments, the plasma kallikrein binding antibody is a monoclonal antibody.

In one aspect, the disclosure features an antibody (e.g., an isolated antibody) that binds to a component of the contact activation system (e.g., human plasma kallikrein antibody and/or murine kallikrein antibody) and includes at least one immunoglobulin variable region. For example, the antibody includes a heavy chain (HC) immunoglobulin variable domain sequence and/or a light chain (LC) immunoglobulin variable domain sequence. In one embodiment, the antibody binds to and inhibits plasma kallikrein, e.g., human plasma kallikrein and/or murine kallikrein.

In some embodiments, the antibodies described herein have the same CDR sequences as lanadelumab-flyo (TAKHZYROⓇ), also referred to as lanadelumab, DX-2930 or SHP463, e.g., heavy chain CDR sequences set forth as SEQ ID NOs: 5-7 and light chain CDR sequences set forth as SEQ ID NOs: 8-10. In some embodiments, the antibody comprises the same CDR sequences as lanadelumab and a LC immunoglobulin variable domain sequence that is at least 85, 88, 89, 90, 91, 92, 93, 94, 95, 96, 97, 98, 99, or 100% identical to a LC variable domain described herein (e.g., overall or in framework regions). In some embodiments, the antibody comprises the same CDR sequences as lanadelumab and an HC immunoglobulin variable domain sequence that is at least 85, 88, 89, 90, 91, 92, 93, 94, 95, 96, 97, 98, 99, or 100% identical to a HC variable domain described herein (e.g., overall or in framework regions). In some embodiments, the antibody comprises the same CDR sequences as lanadelumab and LC sequence that is at least 85, 88, 89, 90, 91, 92, 93, 94, 95, 96, 97, 98, 99, or 100% identical to a LC sequence described herein (e.g., overall or in framework regions). In some embodiments, the antibody comprises the same CDR sequences as lanadelumab and a HC sequence that is at least 85, 88, 89, 90, 91, 92, 93, 94, 95, 96, 97, 98, 99, or 100% identical to a HC sequence described herein (e.g., overall or in framework regions).

The plasma kallikrein antibody may be an isolated antibody (e.g., at least 70, 80, 90, 95, or 99% free of other proteins). In some embodiments, the plasma kallikrein antibody, or composition thereof, is isolated from antibody cleavage fragments (e.g., lanadelumab) that are inactive or partially active (e.g., bind plasma kallikrein with a K_{i,} ₐₚₚ of 5000 nM or greater) compared to the plasma kallikrein binding antibody. For example, the plasma kallikrein antibody is at least 70% free of such antibody cleavage fragments; in other embodiments the binding antibody is at least 80%, at least 90%, at least 95%, at least 99% or even 100% free from antibody cleavage fragments that are inactive or partially active.

The plasma kallikrein antibody may additionally inhibit plasma kallikrein, e.g., human plasma kallikrein. In some embodiments, the plasma kallikrein antibody does not bind prekallikrein (e.g., human prekallikrein and/or murine prekallikrein), but binds to the active form of plasma kallikrein (e.g., human plasma kallikrein and/or murine kallikrein).

In certain embodiments, the antibody binds at or near the active site of the catalytic domain of plasma kallikrein, or a fragment thereof, or binds an epitope that overlaps with the active site of plasma kallikrein.

The antibody can bind to plasma kallikrein, e.g., human plasma kallikrein, with a binding affinity of at least 10⁵, 10⁶, 10⁷, 10⁸, 10⁹, 10¹⁰ and 10¹¹ M⁻¹. In one embodiment, the antibody binds to human plasma kallikrein with a K_{off} slower than 1 × 10⁻³, 5 × 10⁻⁴ s⁻¹, or 1 × 10⁻⁴ s⁻¹. In one embodiment, the antibody binds to human plasma kallikrein with a Kₒₙ faster than 1 × 10², 1 × 10³, or 5 × 10³ M⁻¹s⁻¹. In one embodiment, the antibody binds to plasma kallikrein, but does not bind to tissue kallikrein and/or plasma prekallikrein (e.g., the antibody binds to tissue kallikrein and/or plasma prekallikrein less effectively (e.g., 5-, 10-, 50-, 100-, or 1000-fold less or not at all, e.g., as compared to a negative control) than it binds to plasma kallikrein.

In one embodiment, the antibody inhibits human plasma kallikrein activity, e.g., with a Kᵢ of less than 10⁻⁵, 10⁻⁶, 10⁻⁷, 10⁻⁸, 10⁻⁹, and 10⁻¹⁰ M. The antibody can have, for example, an IC₅₀ of less than 100 nM, 10 nM, 1, 0.5, or 0.2 nM. For example, the antibody may modulate plasma kallikrein activity, as well as the production of Factor XIIa (e.g., from Factor XII) and/or bradykinin (e.g., from high-molecular-weight kininogen (HMWK)). The antibody may inhibit plasma kallikrein activity, and/or the production of Factor XIIa (e.g., from Factor XII) and/or bradykinin (e.g., from high-molecular-weight kininogen (HMWK)). The affinity of the antibody for human plasma kallikrein can be characterized by a K_{D} of less than 100 nm, less than 10 nM, less than 5 nM, less than 1 nM, less than 0.5 nM. In one embodiment, the antibody inhibits plasma kallikrein, but does not inhibit tissue kallikrein (e.g., the antibody inhibits tissue kallikrein less effectively (e.g., 5-, 10-, 50-, 100-, or 1000-fold less or not at all), e.g., as compared to a negative control) than it inhibits plasma kallikrein.

In some embodiments, the antibody has an apparent inhibition constant (K_{i,app}) of less than 1000, 500, 100, 5, 1, 0.5 or 0.2 nM.

Plasma kallikrein binding antibodies may have their HC and LC variable domain sequences included in a single polypeptide (e.g., scFv), or on different polypeptides (e.g., IgG or Fab).

In one embodiment, the HC and LC variable domain sequences are components of the same polypeptide chain. In another, the HC and LC variable domain sequences are components of different polypeptide chains. For example, the antibody is an IgG, e.g., IgG1, IgG2, IgG3, or IgG4. The antibody can be a soluble Fab. In other implementations the antibody includes a Fab2′, scFv, minibody, scFv::Fc fusion, Fab::HSA fusion, HSA::Fab fusion, Fab::HSA::Fab fusion, or other molecule that comprises the antigen combining site of one of the binding proteins herein. The VH and VL regions of these Fabs can be provided as IgG, Fab, Fab2, Fab2′, scFv, PEGylated Fab, PEGylated scFv, PEGylated Fab2, VH::CH1::HSA+LC, HSA::VH::CH1+LC, LC::HSA + VH::CH1, HSA::LC + VH::CH1, or other appropriate construction.

In one embodiment, the antibody is a human or humanized antibody or is non-immunogenic in a human. For example, the antibody includes one or more human antibody framework regions, e.g., all human framework regions, or framework regions at least 85, 88, 89, 90, 91, 92, 93, 94, 95, 96, 97, 98, 99% identical to human framework regions. In one embodiment, the antibody includes a human Fc domain, or an Fc domain that is at least 95, 96, 97, 98, or 99% identical to a human Fc domain.

In one embodiment, the antibody is a primate or primatized antibody or is non-immunogenic in a human. For example, the antibody includes one or more primate antibody framework regions, e.g., all primate framework regions, or framework regions at least 85, 88, 89, 90, 91, 92, 93, 94, 95, 96, 97, 98, 99% identical to primate framework regions. In one embodiment, the antibody includes a primate Fc domain, or an Fc domain that is at least 95, 96, 97, 98, or 99% identical to a primate Fc domain. “Primate” includes humans (Homo sapiens), chimpanzees (Pan troglodytes and Pan paniscus (bonobos)), gorillas (Gorilla gorilla), gibbons, monkeys, lemurs, aye-ayes (Daubentonia madagascariensis), and tarsiers. In some embodiments, the affinity of the primate antibody for human plasma kallikrein is characterized by a K_{D} of less than 1000, 500, 100, 10, 5, 1, 0.5 nM, e.g., less than 10 nM, less than 1 nM, or less than 0.5 nM.

In certain embodiments, the antibody includes no sequences from mice or rabbits (e.g., is not a murine or rabbit antibody).

In some embodiments, the antibody used in the methods described herein may be lanadelumab as described herein or a functional variant thereof.

In one example, a functional variant of lanadelumab comprises the same complementary determining regions (CDRs) as lanadelumab. In another example, the functional variants of lanadelumab may contain one or more mutations (e.g., conservative substitutions) in the FRs of either the V_{H} or the V_{L} as compared to those in the V_{H} and V_{L} of lanadelumab. Preferably, such mutations do not occur at residues which are predicted to interact with one or more of the CDRs, which can be determined by routine technology. In other embodiments, the functional variants described herein contain one or more mutations (e.g., 1, 2, or 3) within one or more of the CDR regions of lanadelumab. Preferably, such functional variants retain the same regions/residues responsible for antigen-binding as the parent. In yet other embodiments, a functional variant of lanadelumab may comprise a V_{H} chain that comprises an amino acid sequence at least 85% (e.g., 90%, 92%, 94%, 95%, 96%, 97%, 98%, or 99%) identical to that of the V_{H} of lanadelumab and/or a V_{L} chain that has an amino acid sequence at least 85% (e.g., 90%, 92%, 94%, 95%, 96%, 97%, 98%, or 99%) identical to that of the V_{L} of lanadelumab. These variants are capable of binding to the active form of plasma kallikrein and preferably do not bind to prekallikrein.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the antibody used in the methods and compositions described herein may be lanadelumab. The heavy and light chain full and variable sequences for lanadelumab are provided below, with signal sequences in italics. The CDRs are boldfaced and underlined.

Lanadelumab Heavy Chain Amino Acid Sequence (451 amino acids, 49439.02 Da)

MGWSCILFLVATATGAHSEVQLLESGGGLVQPGGSLRLSCAASGFTFSHYIMMWVRQ
APGKGLEWVSGIYSSGGITVYADSVKGRFTISRDNSKNTLYLQMNSLRAEDTAVYY
CAYRRIGVPRRDEFDIWGQGTMVTVSSASTKGPSVFPLAPSSKSTSGGTAALGCLV
KDYFPEPVTVSWNSGALTSGVHTFPAVLQSSGLYSLSSVVTVPSSSLGTQTYICNVNH
KPSNTKVDKRVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNG
KEYKCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFSCSVMH
EALHNHYTQKSLSLSPG (SEQ ID NO: 1)

Lanadelumab Light Chain Amino Acid Sequence (213 amino acids, 23419.08 Da)

MGWSCILFLVATATGAHSDIQMTQSPSTLSASVGDRVTITCRASQSISSWLAWYQQKP
GKAPKLLIYKASTLESGVPSRFSGSGSGTEFTLTISSLQPDDFATYYCQQYNTYWTF
GQGTKVEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRG
EC (SEQ ID NO: 2)

Lanadelumab Heavy Chain Amino Acid Sequence (without signal sequence)

EVQLLESGGGLVQPGGSLRLSCAASGFTFSHYIMMWVRQAPGKGLEWVSGIYSSG
GITVYADSVKGRFTISRDNSKNTLYLQMNSLRAEDTAVYYCAYRRIGVPRRDEFDI
WGQGTMVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKRVEPKSC
DKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPG
(SEQ ID NO: 11)

Lanadelumab Light Chain Amino Acid Sequence (without signal sequence)

DIQMTQSPSTLSASVGDRVTITCRASQSISSWLAWYQQKPGKAPKLLIYKASTLESG
VPSRFSGSGSGTEFTLTISSLQPDDFATYYCQQYNTYWTFGQGTKVEIKRTVAAPSV
FIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC (SEQ ID NO: 12)

Lanadelumab Heavy Chain Variable Domain Amino Acid Sequence

EVQLLESGGGLVQPGGSLRLSCAASGFTFSHYIMMWVRQAPGKGLEWVSGIYSSG
GITVYADSVKGRFTISRDNSKNTLYLQMNSLRAEDTAVYYCAYRRIGVPRRDEFDI
WGQGTMVTVSS (SEQ ID NO: 3)

Lanadelumab Light Chain Variable Domain Amino Acid Sequence

DIQMTQSPSTLSASVGDRVTITCRASQSISSWLAWYQQKPGKAPKLLIYKASTLESG
VPSRFSGSGSGTEFTLTISSLQPDDFATYYCQQYNTYWTFGQGTKVEIK (SEQ ID NO:
4)

TABLE 1

CDRs for DX-2930

| | |
|---|---|
| CDR | Amino acid sequence |
| Heavy chain CDR1 | HYIMM (SEQ ID NO: 5) |
| Heavy chain CDR2 | GIYSSGGITVYADSVKG (SEQ ID NO: 6) |
| Heavy chain CDR3 | RRIGVPRRDEFDI (SEQ ID NO: 7) |
| Light chain CDR1 | RASQSISSWLA (SEQ ID NO: 8) |
| Light chain CDR2 | KASTLES (SEQ ID NO: 9) |
| Light chain CDR3 | QQYNTYWT (SEQ ID NO: 10) |

### Antibody Preparation

An antibody as described herein (e.g., lanadelumab) can be made by any method known in the art. See, for example, Harlow and Lane, (1988) Antibodies: A Laboratory Manual, Cold Spring Harbor Laboratory, New York and Greenfield, (2013) Antibodies: A Laboratory Manual, Second edition, Cold Spring Harbor Laboratory Press.

The sequence encoding the antibody of interest, e.g., lanadelumab, may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy in inhibiting the activity of PKal. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse^{RTM} from Amgen, Inc. (Fremont, Calif.) and HuMAb-Mouse^{RTM} and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Antigen-binding fragments of an intact antibody (full-length antibody) can be prepared via routine methods. For example, F(ab′)₂ fragments can be produced by pepsin digestion of an antibody molecule, and Fab fragments that can be generated by reducing the disulfide bridges of F(ab′)₂ fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated or synthesized. The DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage or yeast scFv library and scFv clones specific to a PKal can be identified from the library following routine procedures. Positive clones can be subjected to further screening to identify those that inhibits PKal activity.

Some antibodies, e.g., Fabs, can be produced in bacterial cells, e.g., E. coli cells (see e.g., Nadkarni, A. et al., 2007 Protein Expr Purif 52(1):219-29). For example, if the Fab is encoded by sequences in a phage display vector that includes a suppressible stop codon between the display entity and a bacteriophage protein (or fragment thereof), the vector nucleic acid can be transferred into a bacterial cell that cannot suppress a stop codon. In this case, the Fab is not fused to the gene III protein and is secreted into the periplasm and/or media.

Antibodies can also be produced in eukaryotic cells. In one embodiment, the antibodies (e.g., scFv’s) are expressed in a yeast cell such as Pichia (see, e.g., Powers et al., 2001, J. Immunol. Methods. 251:123-35; Schoonooghe S. et al., 2009 BMC Biotechnol. 9:70; Abdel-Salam, HA. et al., 2001 Appl Microbiol Biotechnol 56(1-2):157-64; Takahashi K. et al., 2000 Biosci Biotechnol Biochem 64(10):2138-44; Edqvist, J. et al., 1991 J Biotechnol 20(3):291-300), Hanseula, or Saccharomyces. One of skill in the art can optimize antibody production in yeast by optimizing, for example, oxygen conditions (see e.g., Baumann K., et al. 2010 BMC Syst. Biol. 4:141), osmolarity (see e.g., Dragosits, M. et al., 2010 BMC Genomics 11:207), temperature (see e.g., Dragosits, M. et al., 2009 J Proteome Res. 8(3):1380-92), fermentation conditions (see e.g., Ning, D. et al. 2005 J. Biochem. and Mol. Biol. 38(3): 294-299), strain of yeast (see e.g., Kozyr, AV et al. 2004 Mol Biol (Mosk) 38(6):1067-75; Horwitz, AH. et al., 1988 Proc Natl Acad Sci USA 85(22):8678-82; Bowdish, K. et al. 1991 J Biol Chem 266(18):11901-8), overexpression of proteins to enhance antibody production (see e.g., Gasser, B. et al., 2006 Biotechol. Bioeng. 94(2):353-61), level of acidity of the culture (see e.g., Kobayashi H., et al., 1997 FEMS Microbiol Lett 152(2):235-42), concentrations of substrates and/or ions (see e.g., Ko JH. et al., 2996 Appl Biochem Biotechnol 60(1):41-8). In addition, yeast systems can be used to produce antibodies with an extended half-life (see e.g., Smith, BJ. et al. 2001 Bioconjug Chem 12(5):750-756).

In one preferred embodiment, antibodies are produced in mammalian cells. Preferred mammalian host cells for expressing the clone antibodies or antigen-binding fragments thereof include Chinese Hamster Ovary (CHO cells) (including dhfr- CHO cells, described in Urlaub and Chasin, 1980, Proc. Natl. Acad. Sci. USA 77:4216-4220, used with a DHFR selectable marker, e.g., as described in Kaufman and Sharp, 1982, Mol. Biol. 159:601 621), lymphocytic cell lines, e.g., NS0 myeloma cells and SP2 cells, COS cells, HEK293T cells (J. Immunol. Methods (2004) 289(1-2):65-80), and a cell from a transgenic animal, e.g., a transgenic mammal. For example, the cell is a mammary epithelial cell.

In some embodiments, plasma kallikrein binding antibodies are produced in a plant or cell-free based system (see e.g., Galeffi, P., et al., 2006 J Transl Med 4:39).

In addition to the nucleic acid sequence encoding the diversified immunoglobulin domain, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216; 4,634,665; and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

In an exemplary system for recombinant expression of an antibody, or antigen-binding portion thereof, a recombinant expression vector encoding both the antibody heavy chain and the antibody light chain is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. For example, some antibodies can be isolated by affinity chromatography with a Protein A or Protein G coupled matrix.

For antibodies that include an Fc domain, the antibody production system may produce antibodies in which the Fc region is glycosylated. For example, the Fc domain of IgG molecules is glycosylated at asparagine 297 in the CH2 domain. This asparagine is the site for modification with biantennary-type oligosaccharides. It has been demonstrated that this glycosylation is required for effector functions mediated by Fcγ receptors and complement C1q (Burton and Woof, 1992, Adv. Immunol. 51:1-84; Jefferis et al., 1998, Immunol. Rev. 163:59-76). In one embodiment, the Fc domain is produced in a mammalian expression system that appropriately glycosylates the residue corresponding to asparagine 297. The Fc domain can also include other eukaryotic post-translational modifications.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly.

### Treatment of Acute Respiratory Distress Syndrome (ARDS)

The present disclosure provides methods for treating acute respiratory distress syndrome comprising administering an inhibitor of the contact activation pathway. Acute respiratory distress syndrome (ARDS) is a life-threatening condition that is induced by an inflammatory reaction following lung trauma. Pulmonary inflammation leads to immune responses that weaken blood vessels and cause fluid leakage into alveoli, which decreases oxygen supply, resulting in respiratory distress. In general, ARDS onset may be associated with a respiratory viral infection, a blood infection, inhalation of toxic substances, an injury to the chest or head, or an overdose of sedatives or tricyclic antidepressants.

ARDS may be categorized as direct ARDS or indirect ARDS. Direct ARDS is caused by injury from an external source to alveolar epithelium leading to inflammation and intra-alveolar edema, whereas indirect ARDS is caused by systemic pathology that diffusely damages the vascular endothelium (Shaver et al., Clin Chest Med. (2014) 35(4): 639-653).

Instances of direct ARDS may be associated with inhalation of toxic substances, such as fine particulate matter from industrial activity, automotive exhaust, pollutants from wildfires, smoke related to cigarettes, cigars, pipe tobacco, hookah, and electronic smoking devices (e.g., e-cigarettes, e-cigs, mods, e-hookahs, vapes); pneumonia; respiratory viral infection; aspiration; near drowning; receiving an injury to the chest or head; and chronic alcohol use (see, e.g., Lin et al. Int J. Biol. Sci. (2018) 14(3): 253-265; Clay and Muller, Recent Increases in Air Pollution: Evidence and Implications for Mortality, NBER Working Paper No. 26381 (2019); “Acute Respiratory Distress Syndrome,” Healthline, Accessed Mar. 25, 2020).

Indirect ARDS may be associated with sepsis-induced immune response resulting in inflammation and subsequent lung damage, transfusion, drug overdose such as sedatives and tricyclic antidepressants (see, e.g., Shaver and Bastarache, Clin Chest Med. (2014) 35(4): 639-653; Sepsis.org, “ARDS,” Accessed Mar. 25, 2020; “Acute Respiratory Distress Syndrome”, Healthline, Accessed Mar. 25, 2020).

The progression of ARDS can be divided into the acute phase, the proliferative phase, and the fibrotic phase. The acute phase typically lasts about 4-7 days and is characterized by damage to the endothelium and epithelium, and pulmonary edema. The proliferative phase typically lasts from about days 7-21 and is characterized by early stage fibrosis in collagen fibers and alveolar epithelial cells. The fibrotic phase typically extends from day 21 onward and is characterized by increased collagen deposition leading to pulmonary fibrosis and a compromised alveolar architecture.

Symptoms of ARDS include shortness of breath, low blood oxygen levels, fast heart rate (e.g., greater than or equal to 100 beats per minute at rest), coughing that produces phlegm, blue fingernails or blue tone to the skin or lips, fatigue, fever, crackling sound in the lungs, chest pain upon breathing deeply, low blood pressure (e.g., lower than 90 mm Hg systolic or 60 mM Hg diastolic), or confusion, or any combination thereof (“Acute Respiratory Distress Syndrome,” National Heart, Lung, and Blood Institute, Accessed Mar. 30, 2021). Common treatment for ARDS includes ventilator support, oxygen therapy, prone positioning, sedation and medications to prevent movement, fluid management, and, in extreme cases, extracorporeal membrane oxygenation (ECMO) (see, e.g., Lung.org, “Acute Respiratory Distress Syndrome (ARDS),” Accessed Mar. 25, 2020).

Without wishing to be bound by any particular theory, inhibition of the contact activation system (e.g., inhibition of plasma kallikrein) may suppress ARDS progression by reducing inflammation and coagulation leading to fibrin deposition that is mediated by the intrinsic pathway as initiated by the contact system. Elevated levels of plasma kallikrein activity, FXIIa, and bradykinin (BK) have been observed in bronchoalveolar lavage fluid (BALF) from ARDS patients (see, e.g., Hess, et al., Thromb Haemost, (2017) 117(10): 1896-1907), and in plasma from ARDS patients. Elevated levels of C1 esterase inhibitor-Factor XIIa (CI-INH-FXIIa), CI-INH-kallikrein, α2M-kallikrein, and inactive C1-INH, reduced levels of FXII, prekallikrein (PK), and high molecular weight kininogen (HMWK), and increased cleavage of HMWK with subsequent production of bradykinin (BK) have been observed in plasma from patients with ARDS. These observations suggest a role for the contact activation system in ARDS. BK is thought to further contribute to lung inflammation and ARDS by stimulating bronchial epithelial cells, alveolar macrophages, and lung fibroblasts to release pro-inflammatory cytokines and chemokines, including IL-6, IL-8, leukotriene B4, platelet-activating factor, monocyte chemoattractant protein-1, granulocyte and granulocyte-macrophage colony-stimulating factor, and transforming growth factor. In LPS-induced sepsis associated with contact system activation-mediated ARDS, bronchoalveolar lavage fluid total leukocyte count (BALF TLC), an indicator of ARDS, was found to be significantly reduced in rat models pre-treated with PF-04886847, an inhibitor of pKal (see, e.g., Kolte, et al., Cardiovasc Hematol Agents Med Chem (2012) 10(2): 154-166). Additionally, rats with Escherichia coli endotoxin-induced pulmonary vascular injury administered a synthetic plasma kallikrein specific inhibitor (PKSI) were found to have reduced pulmonary vascular injury as well as histological changes compared with control mice (see, e.g., Uchiba, et al., Thromb Haemost, (1997) 78(4): 1209-1214). Genetically modified mice lacking the gene encoding plasma kallikrein exhibited reduced lung tissue injury and coagulation after exposure to fine particulate matter, a model of direct ARDS (see, e.g., Wang, et al., Biochem Biophys Res Commun (2019) 518(3): 409-415).

Further, the severity of ARDS associated with SARS-CoV-2 infection can be linked to activation of bradykinin receptor type 1 (BR1). SARS-CoV-2 virus enters cells after interacting with the cellular receptor ACE2, which is needed to inactivate des-Arg⁹ bradykinin, which is a potent ligand of the BR1. When ACE2 is bound by SARS-CoV-2, it cannot inactivate the ligands of BR1 and the lung environment is prone to local vascular leakage, which may lead to angioedema. This could explain why some SARS-CoV-2 patients report feeling like they are drowning. Angioedema leads to inflammation, which induces more BR1 expression and possibly an antibody-dependent enhancement of local immune cells and proinflammatory cytokines (see, e.g., van de Veerdonk, et al., medRXiv (2020)). Therefore, inhibiting the contact system, particularly upstream of BR1 and BR2, may be an effective strategy for treating ARDS associated with SARS-CoV-2.

### ARDS Associated With Viral Infection

As described herein, ARDS may be associated with viral infection, such as a respiratory viral infection. A respiratory viral infection is a viral infection that infects the respiratory system (e.g., sinuses, throat, trachea, lung, bronchial tubes, bronchioles, alveoli, diaphragm).

Non-limiting examples of viruses that cause respiratory viral infection include Coronaviridae (e.g., Middle East Respiratory System (MERS-CoV), severe acute respiratory system (SARS-CoV), severe acute respiratory system coronavirus 2 (SARS-CoV-2, also referred to as 2019 novel coronavirus (2019-nCoV), COVID-19), 229E, NL63, OC43, and HKU1), Orthomyxoviridae (e.g., Influenza A, Influenza B, Influenza C, Influenza D, Isavirus, Thogotovirus, and Quaranjavirus), Paramyxoviridae (Sendai virus (SeV), human parainfluenza virus 3 (hPIV3)), Picornaviridae (e.g., Rhinovirus A (A1, A2, A7-A13, A15, A16, A18-A25, A29-A34, A36, A38-A41, A43-A47, A49-A51, A53-A68, A71, A7-A77, A78, A80-A82, A85, A88-A90, A94-A96, A98, A100-A103), Rhinovirus B (B3-B6, B14, B17, B26, B27, B35, B37, B42, B48, B52, B69, B70, B72, B79, B83, B84, B86, B9-B93, B97, B99), Rhinovirus C (C1-C51)), Adenoviridae (e.g., Human adenovirus B3, B7, B11, B14, B16, B21, B34, B35, B50, B55, Human adenovirus C1, C2, C5, C6, C57), and Parvoviridae (e.g., Bocaparvovirus, Dependoparvovirus, Erythroparvovirus, Protoparvovirus, Tetraparvovirus) (“Human coronavirus types,” Center for Disease Control and Prevention; Nichols, et al. Clin Microbiol Rev (2008) 21: 274-290).

Symptoms associated with respiratory viral infection may include fever, cough, fatigue, sputum production, loss of smell, loss of taste, shortness of breath, muscle or joint pain, sore throat, headache, chills, nausea or vomiting, nasal congestion, diarrhea, haemoptysis, and/or conjunctival congestion.

In some embodiments, the subject has a respiratory viral infection with a coronavirus. In some embodiments, the coronavirus is SARS-CoV-2. Subjects who are experiencing or have experienced a respiratory infection with SARS-CoV-2 may be at risk for developing ARDS and death from respiratory failure (see, e.g., Kaul, Daniel, JAMA Inter Med (2020)). SARS-CoV-2 is a coronavirus belonging to the genus Betacoronavirus. Coronaviruses are single-stranded RNA viruses that may be able to cross species barriers from animals to humans. Although the initiation of SARS-CoV-2 infection in humans is currently unknown, it is speculated that it has an animal origin (see, e.g., Cascella, et al., Features, Evaluation, and Treatment of Coronvavirus (COVID-19) (2020)).

The SARS-CoV-2 virus is round, elliptic, or pleomorphic with a diameter of approximately 60-140 nm. It is sensitive to ultraviolet rays, heat, lipid solvents containing ether (75%), ethanol, chlorine-containing disinfectant (except chlorhexidine), peroxyacetic acid, and chloroforms. Symptoms of SARS-CoV-2 infection typically appear 2-14 days after exposure and include dry cough, fever, fatigue, difficulty breathing, persistent pain or pressure in the chest, new confusion or inability to arouse, bluish lips or face, sudden loss of smell and/or taste, loss of appetite, headache, and sputum production (see, e.g., “Coronavirus Disease 2019 (COVID-19),” Centers for Disease Control and Prevention, Accessed Mar. 25, 2020; “There’s a new symptom of coronavirus, doctors say: Sudden loss of smell or taste”, USA Today, Accessed Mar. 25, 2020; “Coronavirus disease 2019 (COVID-19);” UpToDate, Accessed Mar. 25, 2020).

Laboratory testing for SARS-CoV-2 infection includes methods of detecting the presence of the virus, viral particles, and/or immune responses associated with the viral infection (e.g., antibodies specific to a virus, such as SARS-CoV-2). Example methods for detecting the presence of a virus, such as the SARS-CoV-2 virus, may employ real time reverse transcription polymerase chain reaction (rRT-PCR) and/or use antibodies that specifically bind to the virus. In some embodiments, the method for detecting the presence of the virus, such as the SARS-CoV-2 virus, involves use of antibodies that bind the nucleocapsid protein of the virus, such as the SARS-CoV-2 virus (“Real-time RT-PCR Panel for Detection 2019-nCoV,” Centers for Disease Control and Prevention, Accessed Mar. 25, 2020; “Catching Virus Fast! Academia Sinica discovered useful antibodies for developing rapid immune based test kit of SARS-CoV-2 coronavirus” Sinica.edu.tw, Mar. 9, 2020).

Treatment of severe ARDS associated with respiratory infection with SARS-CoV-2 is a significant challenge. World Health Organization (WHO) guidelines recommend offering ECMO therapy, however complex therapies such as ECMO also present challenges such as limited resources and training of personnel (see, e.g., Ramanathan, et al., Lancet Respir Med (2020) S2213-2600).

### Pneumonia Associated With Respiratory Viral Infection

As described herein, pneumonia may be associated with a viral infection, such as a respiratory viral infection. A respiratory viral infection is a viral infection that infects the respiratory system (e.g., sinuses, throat, trachea, lung, bronchial tubes, bronchioles, alveoli, diaphragm). In some embodiments, the respiratory viral infection is infection with SARS- CoV-2.

Pneumonia is infection of one or both lungs, which may be caused by bacteria, viruses, and/or fungi. A pneumonia infection causes inflammation in the alveoli of the lungs, causing them to fill with fluid or pus and resulting in difficulty breathing. Pneumonia can be life-threatening in any subject, but may be especially dangerous to infants, children, adults over 65 years old, and subjects with pre-existing conditions (e.g., diabetes, heart disease, chronic obstructive pulmonary disease, emphysema, lung cancer).

Symptoms of pneumonia include, but are not limited to phlegm production, pus production, fever, chills, difficulty breathing, and coughing. Antibiotics may be used to treat pneumonia caused by bacteria, and antifungals may be used to treat pneumonia caused by fungi. Some bacteria-caused (e.g., Streptococcus pneumoniae) pneumonias are preventable by vaccine (e.g., Prevnar 13® (Pfizer) and Pneumovax23Ⓡ (Merck)).

Pneumonia may also occur in combination with (e.g., associated with) a respiratory viral infection. In some embodiments, the pneumonia caused by a viral infection. In some embodiments, the respiratory viral infection is an invention with a Coronaviridae virus. In some embodiments, the respiratory viral infection is a SARS-CoV-2 infection, resulting in COVID-19. Pneumonia may also occur in combination with ARDS in a subject having COVID-19. Non-limiting examples of symptoms associated with pneumonia in subjects having COVID-19 include rapid heartbeat, shortness of breath or breathlessness, rapid breathing, dizziness, and heavy sweating (see, e.g. “Coronavirus and pneumonia,” WebMD, https://www.webmd.com/lung/covid-and-pneumonia#1).

Vaccines currently available do not protect against pneumonia associated with SARS-CoV-2 (COVID-19). Current treatments for pneumonia in subjects having COVID-19 are directed to managing the symptoms associated with the viral infection. Subjects with mild to moderate pneumonia associated with SARS-CoV-2 (COVID-19) infection are advised to rest, drink fluids, and isolate until SARS-CoV-2 (COVID-19) tests are negative. Subjects with severe pneumonia associated with SARS-CoV-2 (COVID-19) infection may be hospitalized and receive intravenous (IV) fluids, ventilator, and/or oxygen therapy (see, e.g., “What is the relationship between pneumonia and COVID-19?,” Medical News Today, https://www.medicalnewstoday.com/articles/pneumonia-and-covid-19).

Pneumonia associated with SARS-CoV-2 (COVID-19) may present with different clinical symptoms than pneumonia not associated with SARS-CoV-2 (COVID-19). In particular, pneumonia associated with SARS-CoV-2 (COVID-19) is characterized by a dissociation between the severity of hypoxemia (low oxygen in the blood) and the maintenance of respiratory mechanics, with a median respiratory system compliance that is usually around 50 mL/cm H₂O. There are at least two different types of pneumonia associated with SARS-CoV-2 (COVID-19) referred to as Type 1 and Type 2 pneumonia, which are clearly distinguishable by computed tomography (CT) scan. Type 1 pneumonia associated with SARS-CoV-2 (COVID-19) is characterized by high pulmonary compliance and isolated viral pneumonia, and Type 2 pneumonia associated with SARS-CoV-2 (COVID) is characterized with decreased pulmonary compliance and severe ARDS (see, e.g., Gattinoni et al., “COVID-19 pneumonia: ARDS or not,?” Crit. Care, 2020, 24: 154).

Pulmonary inflammation, such as occurs in pneumonia associated with COVID-19, leads to immune responses that weaken blood vessels and cause edema in alveoli, which decreases oxygen supply. When lung edema reaches a certain magnitude, the gas volume in the lung decreases, resulting in increasingly labored breathing and worsening pneumonia associated with SARS-CoV-2 (COVID-19) (see, e.g., Gattinoni et al., “COVID-19 pneumonia: different respiratory treatments for different phenotypes?,” Intensive Care Medicine, 2020. https://doi.org/10.1007/s00134-020-06033-2). Additionally, plasma kallikrein (pKal) is elevated in bronchoalveolar lavage fluid in subjects with acute pneumonia (see, e.g, Zhang et al., “Kinin Generation in Actue Pneumonia and Chronic Bronchitis,” Eur Respir J., 1997, 10(8): 1747-1753). Thus, an agent that reduces or eliminates pulmonary inflammation by inhibiting the contact activation system (e.g., inhibition of plasma kallikrein) may suppress pneumonia associated with SARS-CoV-2 (COVID-19) infection.

### Methods of Treatment With Inhibitors of the Contact Activation System

The disclosure provides methods of treating (e.g., ameliorating, stabilizing, or eliminating one or more symptoms) acute respiratory distress syndrome (ARDS) by administering an inhibitor of the contact activation system described herein (e.g., a plasma kallikrein antibody) to a subject having or suspected of having ARDS. Additionally provided are methods of treating ARDS by administering an inhibitor of the contact activation system described herein (e.g., a plasma kallikrein antibody) (e.g., a therapeutically effective amount of an inhibitor described herein) according to a dosing schedule described herein, or in combination with one or more additional therapeutic agent described herein. The disclosure also provides methods of preventing ARDS or a symptom thereof by administering an inhibitor of the contact activation system described herein (e.g., a plasma kallikrein antibody) described herein (e.g., a prophylactically effective amount of an antibody described herein) to a subject at risk of developing ARDS (e.g., a subject that has had or is suspected of having an experience associated with ARDS (e.g., a viral respiratory infection (e.g., SARS-CoV-2 infection)). Also provided herein are methods of reducing and/or preventing thrombosis associated with extracorporeal membrane oxygenation (ECMO) in a subject having ARDS.

In some embodiments, the subject is a human patient that has ARDS. In some embodiments, the subject is a human patient that has one or more symptoms associated with ARDS. In some examples, the subject may be a human patient who has no ARDS symptoms at the time of the treatment but has had or is suspected of having an experience associated with ARDS. In some embodiments, the subject has had or is suspected of having a respiratory viral infection, a blood infection, pancreatitis, inhaled toxic substances, an injury to the chest or head, or an overdose of sedatives or tricyclic antidepressants.

In some embodiments, the subject has one or more symptoms of a respiratory viral infection, such as fever, cough, fatigue, sputum production, loss of smell, loss of taste, shortness of breath, muscle or joint pain, sore throat, headache, chills, nausea or vomiting, nasal congestion, diarrhea, haemoptysis, and/or conjunctival congestion.

In some embodiments, the subject has a respiratory viral infection, such as an infection with SARS-CoV-2. In some embodiments, the subject has a confirmed diagnosis of SARS-CoV-2 (e.g., has received a positive result for SARS-CoV-2 in one or more diagnostic detection methods). In some embodiments, the subject has not had confirmed diagnosis for SARS-CoV-2 but is suspected of having an infection with SARS-CoV-2 (e.g., has experienced or is experiencing one or more symptoms associated with infection with SARS-CoV-2).

Also within the scope are subjects having a respiratory viral infection are subjects who previously had a respiratory viral infection, such as an infection with SARS-CoV-2, but no longer have the infection (e.g., the virus is no longer detected). In some embodiments, the subject has antibodies against a virus (e.g., SARS-CoV-2). Antibodies against a virus (e.g., SARS-CoV-2) may be produced following vaccination of the subject or the subject having previously been infected with the virus (e.g., the virus itself is no longer detected). In some embodiments, the subject has been vaccinated against SARS-CoV-2 virus. The subject may be vaccinated with any SARS-CoV-2 vaccine known in the art. Non-limiting examples of SARS-CoV-2 vaccines include mRNA-1273 (Moderna/National Institute of Allergy and Infectious Diseases), BNT162b2 (Pfizer), AZD1222 (AstraZeneca/Oxford), Ad26.COV2.S (Janssen Pharmaceutical), CoronoVac (Sinovac Research and Development), Gam-COVID-Vac (Gamaleya Research Institute), and SARS-CoV-2 rS (Novavax), CVnCoV (CureVac AG) (“Coronavirus disease (COVID-19): Vaccines,” World Health Organization, accessed Mar. 30, 2021)

In some embodiments, the subject may be identified as in need of treatment with an inhibitor of the contact activation system described herein if the subject has one or more symptoms associated with ARDS. In some embodiments, the subject has respiratory distress (e.g., resting breath rate greater than or equal to 30 breaths per minute), fast heart rate (e.g., greater than or equal to 100 beats per minute at rest), coughing that produces phlegm, blue fingernails or blue tone to the skin or lips, fatigue, fever, crackling sound in the lungs, chest pain upon breathing deeply, low blood pressure (e.g., lower than 90 mm Hg systolic or 60 mM Hg diastolic), or confusion, or any combination thereof. Without wishing to be bound by any particular theory, the subject may have increased contact system activation leading to inflammation or coagulation in the lung (e.g., with elevated levels of plasma kallikrein activity, FXIIa, and/or bradykinin) and causing one or more symptoms associated with ARDS, and inhibiting the contact activation system (e.g., with lanadelumab) may suppress or treat the symptom by reducing the lung inflammation and coagulation that is mediated by contact system activation.

In some embodiments, the subject is using a ventilator. In some embodiments, the subject used a ventilator prior to administration of any of the inhibitors of the contact activation system described herein. In some embodiments, the subject has a peripheral capillary oxygen saturation (SpO₂) level that is less than or equal to 93% at rest. In some embodiments, the subject has a ratio of PaO2 to fraction of inspiration O2 (FiO₂) (SpO₂/FiO₂ ratio) that is less than or equal to 300 mmHg. In some embodiments, the subject has pulmonary inflammation. In some embodiments, the subject has inflammatory exudation or pleural effusion.

In some embodiments, the methods described herein are for reducing and/or preventing thrombosis associated with extracorporeal membrane oxygenation (ECMO) in a subject having ARDS. ECMO is used to support critically ill patients with severe respiratory or cardiac failure, such as in a subject having ARDS. Despite improvement in ECMO technology, thrombosis remains a significant complication, as the interaction between the patient’s blood and the foreign surface of the ECMO circuit can activate the contact activation system (see, e.g., Dalton, et al., Pediatr Crit Care Med. (2015), 16(2): 167-174). “Thrombosis,” as used herein, is the formation of a blood clot, known as a thrombus, within a blood vessel that prevents blood from flowing normally through the circulatory system. Symptoms of thrombosis include, but are not limited to swelling, pain, redness, warmth to the touch, worsening leg or arm pain when bending the foot or hand, leg cramps, discoloration of skin. Treatments for thrombosis included anticoagulation, thrombolysis, surgery, endovascular treatment, and targeting ischemia/reperfusion injury.

The disclosure also provides methods of treating (e.g., ameliorating, stabilizing, or eliminating one or more symptoms) thrombosis associated with ECMO by administering an inhibitor of the contact activation system described herein (e.g., a plasma kallikrein antibody) to a subject having or suspected of having ARDS. Additionally provided are methods of treating thrombosis associated with ECMO by administering an inhibitor of the contact activation system described herein (e.g., a plasma kallikrein antibody) (e.g., a therapeutically effective amount of an inhibitor described herein) according to a dosing schedule described herein, or in combination with one or more additional therapeutic agent described herein. The disclosure also provides methods of preventing thrombosis associated with ECMO or a symptom thereof by administering an inhibitor of the contact activation system described herein (e.g., a plasma kallikrein antibody) (e.g., a prophylactically effective amount of an antibody described herein) to a subject at risk of developing thrombosis associated with ECMO (e.g., a subject that is having or may have to have ECMO).

In some embodiments, the subject is a human patient that has thrombosis associated with ECMO. In some embodiments, the subject is a human patient that has one or more symptoms associated with thrombosis associated with ECMO. In some examples, the subject may be a human patient who has no thrombosis associated with ECMO symptoms at the time of the treatment but has had or is suspected of having an experience associated with thrombosis associated with ECMO. In some embodiments, the subject has had or is suspected of having a respiratory viral infection, swelling, pain, redness, warmth to the touch, worsening leg or arm pain when bending the foot or hand, leg cramps, and/or discoloration of skin.

In some embodiments, the subject is an immunocompromised subject. As used herein, “immunocompromised” refers to an impaired or weakened immune system. Subjects who are immunocompromised have a reduced ability to fight infections and other diseases, and accordingly may be more susceptible to infection, such as infection with a respiratory virus. A patient may be immunocompromised by certain diseases or conditions, such as acquired immunodeficiency syndrome (AIDS), cancer, diabetes, malnutrition, burns, immune-complex diseases (viral hepatitis) and certain genetic disorders including, but not limited to, hypogammaglobulinemia, agammaglobulinemia, ataxia-telangiectasia, DiGeorge syndrome, Job syndrome, leukocyte adhesion defects, common variable immunodeficiency, severe combined immunodeficiency, Chediak-Higashi syndrome, combined immunodeficiency disease, panhypogammaglobulinemia, Bruton’s disease, selective deficiency of IgA, Wiskott-Aldrich syndrome, autoimmune lymphoproliferative syndrome, autoimmune polyglandular syndrome, BENTA disease, caspase eight deficiency state, CARD9 deficiency, chronic granulomatous disease, common variable immunodeficiency, congenital neutropenia syndrome, CTLA4 deficiency, DOCK8 deficiency, GATA2 deficiency, hyper-immunoglobulin E syndrome, hyper-immunoglobulin M syndrome, interferon gamma deficiency, interleukin 12 deficiency, interleukin 23 deficiency, LRBA deficiency, PI3K kinase disease, STAT3 dominant-negative disease, STAT3 gain-of-function disease, and XMEN disease. A patient may also be immunocompromised as a result of certain medicines or treatments, such as anticancer drugs, radiation therapy, stem cell transplant, or organ transplant. Non-limiting examples of medications that cause immunosuppression include: tacrolimus, cyclosporine, mycophenolate mofetil, mycophenolate sodium, azathioprine, sirolimus, prednisone, prednisolone, methylprednisolone, methotrexate, leflunomide, cyclophosphamide, chlorambucil, nitrogen mustard, dexamethasone, hydrocortisone, mercaptopurine, fluorouracil, dactinomycin, anthracycline, mitomycin C, bleomycin, mithramycin, interferon β, infliximab (RemicadeⓇ), etanercept (EnbrelⓇ), adalimumab (HumiraⓇ), fingolimod, and myriocin. Common symptoms of being immunocompromised include pinkeye, sinus infections, colds, diarrhea, pneumonia, yeast infections, ear infections, meningitis, skin infections, inflammation and infection of internal organs, blood disorders (low platelet counts, anemia), loss of appetite, abdominal cramping, nausea, delayed growth and development.

In some embodiments, the subject is a pediatric subject. In some embodiments, the subject is an adolescent less than 18 years old. In some embodiments, the subject is an adolescent between the ages of 12 and 18 years old. In some embodiments, the subject is between the ages of 19 and 30 years old. In some embodiments, the subject is between the ages of 30 and 50 years old. In some embodiments, the subject is between the ages of 50 and 65 years old. In some embodiments, the subject is a geriatric patient (e.g., more than 65 years old).

In some embodiments, the subject may be defined by gender. For example, in some embodiments, the subject is female. In some embodiments, the subject is male.

Treating includes administering an amount effective to alleviate, relieve, alter, remedy, ameliorate, improve or affect the disorder, the symptoms of ARDS or the predisposition toward the ARDS. The treatment may also delay onset, e.g., prevent onset, or prevent deterioration of a disease or condition.

Methods of administering inhibitors of the contact activation system (e.g., plasma kallikrein antibodies) are also described in “Pharmaceutical Compositions.” Suitable dosages of the antibody used can depend on the age and weight of the subject and the particular drug used.

In some embodiments, the inhibitor of the contact activation system is a plasma kallikrein antibody (e.g., lanadelumab). The antibody can be used as competitive agents to inhibit, reduce an undesirable interaction, e.g., between plasma kallikrein and its substrate (e.g., Factor XII or HMWK). The dose of the antibody can be the amount sufficient to block 90%, 91%, 92%, 93%, 94%, 95%, 96%, 97%, 98% 99%, or 99.9% of the activity of plasma kallikrein in the patient, especially at the site of disease.

In one embodiment, the antibodies are used to inhibit an activity (e.g., inhibit at least one activity of plasma kallikrein, e.g., reduce Factor XIIa and/or bradykinin production) of plasma kallikrein, e.g., in vivo. The binding proteins can be used by themselves or conjugated to an agent, e.g., a cytotoxic drug, cytotoxin enzyme, or radioisotope.

The antibodies can be used directly in vivo to eliminate antigen-expressing cells via natural complement-dependent cytotoxicity (CDC) or antibody dependent cellular cytotoxicity (ADCC). The antibodies described herein can include complement binding effector domain, such as the Fc portions from IgG1, -2, or -3 or corresponding portions of IgM which bind complement. In one embodiment, a population of target cells is ex vivo treated with an antibody described herein and appropriate effector cells. The treatment can be supplemented by the addition of complement or serum containing complement. Further, phagocytosis of target cells coated with an antibody described herein can be improved by binding of complement proteins. In another embodiment target, cells coated with the antibody which includes a complement binding effector domain are lysed by complement.

A therapeutically effective amount of an antibody as described herein, can be administered to a subject having, suspected of having, or at risk for ARDS, thereby treating (e.g., ameliorating or improving a symptom or feature of a disorder, slowing, stabilizing and/or halting disease progression) the disorder.

The inhibitor of the contact activation system described herein can be administered in a therapeutically effective amount. A therapeutically effective amount of an inhibitor of the contact activation system (e.g., plasma kallikrein antibody) is the amount which is effective, upon single or multiple dose administration to a subject, in treating a subject, e.g., curing, alleviating, relieving, or improving at least one symptom of a disorder in a subject to a degree beyond that expected in the absence of such treatment.

Dosage regimens can be adjusted to provide the optimum desired response (e.g., a therapeutic response). For example, a single bolus may be administered, several divided doses may be administered over time or the dose may be proportionally reduced or increased as indicated by the exigencies of the therapeutic situation. In other examples, a bolus may be administered followed by several doses over time or the dose may be proportionally reduced or increased as indicated by the exigencies of the therapeutic situation. In other examples, a dose may be divided into several doses and be administered over time. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

In some embodiments, the inhibitor of the contact activation system is administered in multiple doses. In some embodiments, the therapeutically or prophylactically effective amount of the inhibitor of the contact activation system (e.g., plasma kallikrein antibody) can be about 100 mg - 400 mg. In some embodiments, the therapeutically or prophylactically effective amount of the inhibitor of the activation system (e.g., plasma kallikrein antibody) can be about 150 mg or 300 mg and is administered every day, every two days, every three days, every four days, every five days, every six days, every week, every two weeks, every three weeks, every four weeks, every five weeks, every six weeks, every seven weeks, every eight weeks or longer.

In some embodiments, the therapeutically or prophylactically effective amount of a plasma kallikrein inhibitor (e.g., lanadelumab) can be about 150 mg or 300 mg and is administered to a subject in multiple doses. In some embodiments, the therapeutically or prophylactically effective amount of a plasma kallikrein inhibitor (e.g., lanadelumab) can be about 150 mg or 300 mg and is administered to a subject every day, every two days, every three days, every four days, every five days, every six days, every week, every two weeks, every three weeks, every four weeks, every five weeks, every six weeks, every seven weeks, every eight weeks or longer. In specific examples, the antibody is administered to the subject at about 300 mg every two weeks; or every three days.

In some embodiments, the therapeutically or prophylactically effective amount is administered one time, at least two times, at least three times, at least four times, at least five times, at least six times, at least seven times, at least eight times, at least nine times, at least ten times, at least eleven times, at least twelve time, at least thirteen times, or more.

In any of the embodiments described herein, the timing of the administration of the antibody is approximate and may include the three days prior to and three days following the indicated administration (e.g., administration every two weeks encompasses administration on day 11, day 12, day 13, day 14, day 15, day 16, or day 17, the previous dose being administered on day 1). In any of the embodiments described herein, the timing of the administration of the antibody is approximate and may include one day before and one day following the indicated administration (e.g., administration every three days encompasses administration on day 3, day 4, or day 5, the previous dose being administered on day 1).

The prior administration of one or more additional therapeutic agent can involve the same inhibitor of the contact activation system as described herein (e.g., a plasma kallikrein antibody). In some embodiments, the prior administration of one or more additional therapeutic agent can involve another inhibitor of the contact activation system as described herein (e.g., a C1-inhibitor, a pKal inhibitor, or a bradykinin receptor antagonist).

In some embodiments, the prior administration of one or more additional therapeutic agent can involve treatment of ARDS. In some embodiments, the prior administration of one or more additional therapeutic agent can involve treatment of a viral infection that may be associated ARDS. In some embodiments, the prior administration of one or more additional therapeutic agent can involve treatment of a coronavirus infection (e.g., SARS-CoV-2). In some embodiments, the one or more additional therapeutic is an immunomodulatory agent, such as any of the inhibitors of IL-6R described herein. In some embodiments, the one or more additional therapeutic is an antiviral agent, such as any of the antiviral agents described herein. In some embodiments, the one or more additional therapeutic is an anti-malarial agent, such as any of the anti-malarial agents described herein.

In some embodiments, administering an inhibitor of the contact activation system according to any of the methods described herein results in a reduction of one or more symptoms of ARDS in the subject. In some embodiments, a percent reduction of a reduction of one or more symptoms of ARDS after administering an antibody according to any of the methods described herein may be determined relative to the one or more symptoms of ARDS in subjects who did not receive the inhibitor (e.g., subjects that were administered a placebo, another therapy, or no therapy). In some embodiments, the percent reduction of one or more symptoms of ARDS may be at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% relative to a reduction of one or more symptoms of ARDS in subjects who did not receive the inhibitor (e.g., subjects that were administered a placebo, another therapy, or no therapy).

In some embodiments, the subject has received one or more prior treatment for ARDS, such as ventilator support, oxygen therapy, prone positioning, sedation and medications to prevent movement, fluid management, and, in extreme cases, and/or extracorporeal membrane oxygenation (ECMO).

In some embodiments, the therapeutically effective amount of the antibody (e.g., lanadelumab) can be about 100 mg - 400 mg and is administered to a subject that has received one or more prior treatments for ARDS, every two weeks, every three weeks, every four weeks, every five weeks, every six weeks, every seven weeks, every eight weeks or longer. In some embodiments, the therapeutically effective amount of the antibody (e.g., lanadelumab) can be about 150 mg or 300 mg and is administered to a subject that has received one or more prior treatments for ARDS, every two weeks, every three weeks, every four weeks, every five weeks, every six weeks, every seven weeks, every eight weeks or longer.

Any of the methods described herein may further comprise monitoring the patient for side effects (e.g., elevation of creatine phosphatase levels) and/or inhibition levels of the contact activation system by the inhibitor thereof before and after the treatment or during the course of treatment. If one or more adverse effect is observed, the dose of the inhibitor might be reduced or the treatment might be terminated. If the inhibition level is below a minimum therapeutic level, further doses of the inhibitor might be administered to the patient. Patients may also be evaluated for the generation of antibody against the administered inhibitor; need for a ventilator, number of days on a ventilator, in-hospital time (e.g., number of days spent in the hospital associated with ARDS or a condition associated with ARDS), measure of respiratory distress (e.g., resting breathing rate), peripheral capillary oxygen saturation (SpO₂), partial pressure of oxygen in the alveoli (PaO₂), SpO₂/FiO₂ ratio, fraction of inspired oxygen (FiO₂), pulmonary inflammation (e.g., by chest imaging), pleural effusion, blood pressure, and/or need for vasopressor agent(s).

In some embodiments, the plasma or serum concentration of the inhibitor of the contact activation pathway may be measured during the course of the treatment (e.g., after the initial dosage) for assessing the efficacy of the treatment. If the plasma or serum concentration of the contact activation pathway is lower than about a threshold level, a follow-up dosage may be needed, which may be the same or higher than the initial dosage. The plasma or serum concentration of the contact activation pathway may be measured by determining the protein level of the inhibitor in a plasma or serum sample obtained from the subject, e.g., by an immune assay or MS assay. The plasma or serum concentration of the inhibitor may also be measured by determining the inhibitory level of contact activation system (e.g., plasma kallikrein) in a plasma or serum sample obtained from a subject treated with the inhibitor. Such assays may include a synthetic substrate assay or a Western blot assay for measuring cleaved kininogen.

Alternatively or in addition, the plasma or serum level of creatine kinase and/or one or more coagulation parameters (e.g., activated partial thromboplastin time (aPTT), prothrombin time (PT), bleeding events) can be monitored during the course of the treatment. If the plasma or serum level of creatine kinase is found to elevate during the treatment, the dosage of the inhibitor of the contact activation system may be reduced or the treatment may be terminated. Similarly, if one or more coagulation parameters (other than aPTT, which is known to be prolonged by plasma kallikrein inhibition without impacting normal hemostasis) are found to be significantly affected during the treatment, the dosage of the inhibitor of the contact activation pathway may be modified or the treatment may be terminated.

### Combination Therapies

An inhibitor of the contact activation system as described herein (e.g., a plasma kallikrein antibody, such as lanadelumab) can be administered to the subject in combination with one or more additional therapeutic agents. In some embodiments, the methods described herein involve administering one or more additionally therapeutic agent to the subject. In some embodiments, the subject has been administered one or more additional therapeutic agents prior to administering the inhibitor of the contact activation pathway (e.g., plasma kallikrein antibody).

In some embodiments, the additional therapeutic agent is an immunomodulatory agent, or combination thereof. In some embodiments, the immunomodulatory agent is an inhibitor of the interleukin-6 receptor (IL-6R). IL-6R is a protein complex consisting of an IL-6 receptor subunit (IL-6R) and interleukin 6 signal transducer glycoprotein 130. IL-6 is a cytokine secreted upon infection or trauma to stimulate the immune system response, and is partially responsible for fever and triggering inflammation in many diseases. IL-6 activates target cells via the membrane bound IL-6R. Soluble forms of IL-6R may be found in high amounts in the serum of healthy individuals. Without wishing to be bound by any particular theory, it is generally considered that inhibiting or blocking IL-6R may reduce the proinflammatory properties of IL-6. Non-limiting examples of IL-6R inhibitors include tocilizumab (ActemraⓇ), sarilumab (KevzaraⓇ), levilimab (BCD 089), LMT-28 (Hong, et al. J Immunol (2015) 195(1): 237-245), DTA(A7/sTNFR2), and thalidomide (ThalomidⓇ) (Scavone, et al., British J. Pharmacol. (2020) 177: 4813-4824). In some embodiments, an immunomodulatory agent modulates a different immune system target that is not IL-6. Non-limiting examples of immunomodulatory agents that modulate an immune system target that is not IL-6 include Janus kinase inhibitors (barcitinib (OlumiantⓇ), fedratanib (InrebicⓇ), and ruxolitinib (JakafiⓇ)), inflammation inhibitors (aviptadil (ZyesamiⓇ, RLF-100)); complement inhibitors (eculizumab (SolirisⓇ)), receptor tyrosine kinase inhibitors (sunitinib (SutentⓇ)); (Kim, et al., Scientific Reports (2016) 6: 20150), programmed cell death 1 (PD-1) inhibitors (camrelizumab (SHR-1210)), vascular endothelial growth factor (VEGF) inhibitors (bevacizumab (AvastinⓇ)), and dihydroorotate dehydrogenase inhibitors (leflunomide (AravaⓇ)), (Scavone, et al., British J. Pharmacol. (2020) 177: 4813-4824). In some embodiments, the immunomodulatory agent is tocilizumab (ActemraⓇ). In some embodiments, the immunomodulatory agent is sarilumab (KevzaraⓇ). In some embodiments, the additional therapeutic agent is an antiviral agent, or a combination thereof. An antiviral agent is a compound or molecule that inhibits production of a virus (e.g., viral infection). In some embodiments, the antiviral agent inhibits a viral protein or activity thereof. In some embodiments, the antiviral agent inhibits (targets) the virus at a particular stage of the viral lifecycle (e.g., cellular entry, uncoating, reverse transcription, integration, transcription, translation, protein processing, virion assembly, and/or virion release). In some embodiments, the additional therapeutic agent comprises a combination of an immunomodulatory agent and an antiviral agent.

Non-limiting examples of antiviral agents include adamantane antivirals (amantadine (SymmetrelⓇ), rimantadine (FlumadineⓇ), antiviral boosters (ritonavir (NorvirⓇ), cobicistat (TybostⓇ)), antiviral combinations (abacavir/lamivudine (EpzicomⓇ), cobicistat/elvitegravir/emtricitabine/tenofovir (StribildⓇ), doletegravir/lamivudine (DovatoⓇ), abacavir/lamivudine/zidovudine (TrizivirⓇ), elbasvir/grazoprevir (ZepatierⓇ), efavirenz/emtricitabine/tenofovir (AtiplaⓇ), glecaprevir/pibrentasvir (MavyretⓇ), ledipasvir/sofosbuvir (HarvoniⓇ), emtricitabine/rilpivirine/tenofovir (CompleraⓇ), abacavir/dolutegravir/lamivudine (TriumeqⓇ), sofosbuvir/velpatasvir (EpclusaⓇ), emtricitabine/rilpivirine/tenofovir alafenamide (OdefseyⓇ), cobicistat/darunavir/emtricitabine/tenofovir alafenamide (SymtuzaⓇ), emtricitabine/tenofovir (TruvadaⓇ), bictegravir/emtricitabine/tenofovir alafenamide (BiktarvyⓇ), cobicistat/elvitegravir/emtricitabine/tenofovir alafenamide (GenvoyaⓇ), dolutegravir/rilpivirine (JulucaⓇ) dadabuvir/ombitasvir/paritaprevir/ritonavir (Viekira PakⓇ), lamivudine/zidovudine (CombivirⓇ), emtriciabine/tenofovir alafenamide (DescovyⓇ), cobicistat/darunavir (PrezcobixⓇ), emtricitabine/tenefovir (AccessPak for HIV PEP BasicⓇ), emtricitabine/lopinavir/ritonavir/tenofovir (AccessPak for HIV PEP Expanded with KaletraⓇ), emtricitabine/nelfinavir/tenofovir (AccessPak for HIV PEP Expanded with ViraceptⓇ), lamivudine/tenofovir (CimduoⓇ), doravirine/amivudine/tenofovir (DelstrigoⓇ), atazanavir/cobicistat (EvotazⓇ), efavirenz/lamivudine/tenofovir (SymfiⓇ), ombitasvir/paritaprevir/ritonavir (TechnivieⓇ), lamivudine/tenofovir (TemixysⓇ), dadabuvir/ombitasvir/paritaprevir/ritonavir (Viekira XRⓇ), sofosbuvir/velpatasvir/volxilaprevir (VoseviⓇ)), antiviral interferons (peginterferon alfa-2a (PegasysⓇ), peginterferon alfa-2b (PegIntronⓇ, SylatronⓇ)), chemokine receptor antagonists (maraviroc (SelzentryⓇ)), integrase strand transfer inhibitor (raltegravir (IsentressⓇ), dolutegravir (TivicayⓇ), elvitegravir (VitektaⓇ)), neuraminidase inhibitors (zanamivir (RelenzaⓇ), oseltamivir (TamifluⓇ), peramivir (RapivabⓇ)), non-nucleoside reverse transcriptase inhibitors (NNRTIs) (etravirine (IntelenceⓇ), efavirenz (SustivaⓇ), nevirapine (ViramuneⓇ), rilpivirine (EdurantⓇ), doravirine (PifeltroⓇ), delaviridine (RescriptorⓇ), nevirapine (Viramune XRⓇ)), non-structural protein 5A (NS5A) inhibitors (daclatasvir (DaklinzaⓇ)), nucleoside reverse transcriptase inhibitors (NRTIs) entecavir (BaracludeⓇ), lamivudine (Epivir-HBVⓇ), adefovir (HepseraⓇ), didanosine (VidexⓇ), tenofovir alafenamide (VemlidyⓇ), abacavir (ZiagenⓇ), tenofovir (VireadⓇ), lamivudine (EpivirⓇ), zidovudine (RetrovirⓇ), stavudine (ZeritⓇ), emtrictabine (EmtrivaⓇ), zalcitabine (HividⓇ), telbivudine (TyzekaⓇ), didanosine (Videx ECⓇ)), protease inhibitors (lopinavir, boceprevir (VictrelisⓇ), simeprevir (OlysioⓇ), fosamprenavir (LexivaⓇ), lopinavir/ritonavir (KaletraⓇ), darunavir (PrezistaⓇ), telaprevir (IncivekⓇ), tipranavir (AptivusⓇ), ritonavir (NorvirⓇ), atazanavir (ReyatazⓇ), nelfinavir (ViraceptⓇ), amprenavir (AgeneraseⓇ), indinavir (CrixivanⓇ), saquinavir (FortovaseⓇ), saquinavir (InviraseⓇ)), purine nucleosides (ribavirin (RibasphereⓇ), valacyclovir (ValtrexⓇ), acyclovir (ZoviraxⓇ, SitavigⓇ), famciclovir (FamvirⓇ), ribavirin (CopegusⓇ, ModeribaⓇ), valganciclovir (ValcyteⓇ), ribavirin (RebetolⓇ, RibaPakⓇ, RibaTabⓇ, VirazoleⓇ), ganciclovir (CytoveneⓇ), cidofovir (VistideⓇ)), and miscellaneous antivirals (remedesivir, interferon-beta (IFN-β), umifenovir (ArbidolⓇ), fomivirsen (VitraveneⓇ), sofosbuvir (SovaldiⓇ), baloxavir marboxil (XofluzaⓇ), enfuviritide (FuzeonⓇ), foscarnet (FoscavirⓇ), letermovir (PrevymisⓇ), ibalizumab (TrogarzoⓇ) (“Antiviral Agents”, Drugs.com, Accessed Mar. 25, 2020), favipiravir (AviganⓇ), camostat mesilate/nafamostat, triazavirin (RiamilovirⓇ), azidovudine, danoprevir, meplazumab, and ivermectin (SoolantraⓇ, SkliceⓇ, StromectolⓇ) (Scavone, et al., British J. Pharmacol. (2020) 177: 4813-4824). In some embodiments, the antiviral agent is lopinavir. In some embodiments, the antiviral agent is ritonavir. In some embodiments, the antiviral agent is a combination of lopinavir and ritonavir (KaletraⓇ). In some embodiments, the antiviral agent is interferon-beta. In some embodiments, the antiviral agent is umfenovir. In some embodiments, the antiviral agent is remdesivir.

In some embodiments, the additional therapeutic agent is an anti-malarial agent. Malaria is an infectious disease caused by Plasmodium parasites, which is transmitted by infected female Anopheles mosquitos. An anti-malarial agent is a compound or molecule that is effective in the treatment of malaria and/or effective against Plasmodium parasites or infection with Plasmodium parasites. Anti-malarial agents are generally classified according to their action against different stages of the life cycle of the Plasmodium parasite. Non-limiting examples of anti-viral agents include antimalarial combinations (pyrimethamine/sulfadoxine (FansidarⓇ), artemether/lumefantrine (CoartemⓇ), atovaquone/proguanil (MalaroneⓇ, Malarone PediatricⓇ), antimalarial quinolones (chloroquine (Aralen Phosphate), quinine (QualaquinⓇ), tafenoquine (ArakodaⓇ, KrintafelⓇ), hydroxychloroquine (PlaquenilⓇ), mefloquine (LariamⓇ), and miscellaneous antimalarials (doxycycline (Adoxa TT, Oraxyl, AdoxaⓇ, OraceaⓇ, MonodoxⓇ, Doxy 100Ⓡ, VibramycinⓇ, DoryxⓇ, Adoxa CK, Alodox, Avidoxy, Morgidox, Ocudox, PeriostatⓇ, Bira-Tabs), pyrimethamine (DaraprimⓇ), halofantrine (Halfan) (“Antimalarial Agents”, Drugs.com, Accessed Mar. 25, 2020), and piperaquine (Scavone, et al., British J. Pharmacol. (2020) 177: 4813-4824). In some embodiments, the anti-malarial agent is chloroquine.

In some embodiments, the additional therapeutic agent is an antimetabolite anti-neoplastic agent. An antimetabolite anti-neoplastic agent is a compound or molecule that acts to prevent, inhibit, or halt the development of a neoplasm (e.g., a tumor) by modulating one or more enzymes or reactions that are necessary for DNA synthesis. Non-limiting examples of anti-neoplastic agents include decitabine (DacogenⓇ), fluorouracil (AdrucilⓇ), cladribine (LeustatinⓇ, MavencladⓇ), methotrexate (OtrexupⓇ, TrexallⓇ, RheumatrexⓇ Dose Pack, RasuvoⓇ, XatmepⓇ), mercaptopurine (PurinetholⓇ), pemetrexed (AlimtaⓇ, PemfexyⓇ), gemcitabine (GemzarⓇ, InfugemⓇ), capecitabine (XelodaⓇ), hydroxyurea (HydreaⓇ, DroxiaⓇ, MylocelⓇ, SiklosⓇ), fludarabine (FludaraⓇ, OfortaⓇ), pralatrexate (FolotynⓇ), nelarabine (ArranonⓇ), clofarabine (ClolarⓇ), cytarabine liposomal (DepoCytⓇ), floxuridine (FUDRⓇ), and thioguanine (TabloidⓇ) (“Antimetabolites”, Drugs.com, Accessed Mar. 30, 2021). In some embodiments, the antimetabolite anti-neoplastic agent is decitabine.

One or more additional inhibitors of the contact activation system can be used in combination with an antibody as described herein (e.g., lanadelumab). For example, the combination can result in a lower dose of the inhibitor being needed, such that side effects are reduced. In some embodiments, the combination can result in further reduction or amelioration of one or more symptoms associated with ARDS, for example as compared to use of one agent alone.

In some embodiments, the additional inhibitor of the contact activation system is a C1-inhibitor, a plasma kallikrein inhibitor, or a bradykinin receptor antagonist. In some embodiments, the additional inhibitor of the contact activation system is ecallantide, a C1 esterase inhibitor (e.g., CINRYZE™), a broader spectrum protease inhibitor, such as aprotinin (TRASYLOLⓇ), and/or a bradykinin B2 receptor inhibitor (e.g., icatibant (FIRAZYRⓇ)).

Examples of plasma kallikrein inhibitors that can be used in combination therapy with an inhibitor of the contact activation system, such as plasma kallikrein antibodies described herein include plasma kallikrein inhibitors described in, e.g., PCT Publication No. WO 95/21601 or WO 2003/103475.

The term “combination” refers to the use of the two or more agents or therapies to treat the same patient, wherein the use or action of the agents or therapies overlaps in time. The agents or therapies can be administered at the same time (e.g., as a single formulation that is administered to a patient or as two separate formulations administered concurrently) or sequentially in any order. Sequential administrations are administrations that are given at different times. The time between administration of the one agent and another agent can be minutes, hours, days, or weeks. The use of a plasma kallikrein binding antibody described herein can also be used to reduce the dosage of another therapy, e.g., to reduce the side effects associated with another agent that is being administered. Accordingly, a combination can include administering a second agent at a dosage at least 10, 20, 30, or 50% lower than would be used in the absence of the plasma kallikrein binding antibody. In some embodiments, a subject can be given a C1-inhibitor as a loading intravenous (IV) dose or subcutaneous (SC) dose simultaneously with the first dose of an anti-pKal antibody (e.g., lanadelumab/DX-2930) as described herein. In some embodiments, a subject can be given a C1-inhibitor as a loading IV dose or SC dose simultaneously with a first IV dose of an anti-pKal antibody (e.g., lanadelumab/DX-2930) as described herein. In some embodiments, a subject can be given a C1-inhibitor as a loading IV dose or SC dose simultaneously with a first SC dose of an anti-pKal antibody (e.g., lanadelumab/DX-2930) as described herein. The subject can then continue with the anti-pKal antibody treatment (either IV or SC, without further doses of the C1-inhibitor).

A combination therapy can include administering an agent that reduces the side effects of other therapies. The agent can be an agent that reduces the side effects of a plasma kallikrein associated disease treatment.

### Pharmaceutical Compositions

An inhibitor of the contact activation system as described herein (e.g., plasma kallikrein antibody) can be present in a composition, e.g., a pharmaceutically acceptable composition or pharmaceutical composition. The inhibitor of the contact activation system as described herein (e.g., plasma kallikrein antibody) can be formulated together with a pharmaceutically acceptable carrier.

In some embodiments, about 100 mg to 400 mg of a plasma kallikrein antibody (e.g., lanadelumab) is present in a composition optionally with a pharmaceutically acceptable carrier, e.g., a pharmaceutically acceptable composition or pharmaceutical composition. In some embodiments, the composition comprises about 150 mg or 300 mg of a plasma kallikrein antibody (e.g., lanadelumab), optionally with a pharmaceutically acceptable carrier.

A pharmaceutically acceptable carrier includes any and all solvents, dispersion media, coatings, antibacterial and antifungal agents, isotonic and absorption delaying agents, and the like that are physiologically compatible. Preferably, the carrier is suitable for subcutaneous, intravenous, intramuscular, parenteral, spinal, or epidermal administration (e.g., by injection or infusion), although carriers suitable for inhalation and intranasal administration are also contemplated.

The pharmaceutically acceptable carrier in the pharmaceutical composition described herein may include one or more of a buffering agent, an amino acid, and a tonicity modifier. Any suitable buffering agent or combination of buffering agents may be used in the pharmaceutical composition described herein to maintain or aid in maintaining an appropriate pH of the composition. Non-limiting examples of buffering agents include sodium phosphate, potassium phosphate, citric acid, sodium succinate, histidine, Tris, and sodium acetate. In some embodiments, the buffering agents may be at a concentration of about 5-100 mM, 5-50 mM, 10-50 mM, 15-50 mM, or about 15-40 mM. For example, the one or more buffering agents may be at a concentration of about 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 35 mM, 36 mM, 37 mM, 38 mM, 39 mM, or about 40 mM. In some examples, the pharmaceutically acceptable carrier comprises sodium phosphate and citric acid, which may be at a concentration of about 30 mM and about 19 mM, respectively.

In some embodiments, the pharmaceutically acceptable carrier includes one or more amino acids, which may decrease aggregation of the antibody and/or increase stability of the antibody during storage prior to administration. Exemplary amino acids for use in making the pharmaceutical compositions described herein include, but are not limited to, alanine, arginine, asparagine, aspartic acid, glycine, histidine, lysine, proline, or serine. In some examples, the concentration of the amino acid in the pharmaceutical composition may be about 5-100 mM, 10-90 mM, 20-80 mM, 30-70 mM, 40-60 mM, or about 45-55 mM. In some examples, the concentration of the amino acid (e.g., histidine) may be about 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, or about 60 mM. In one example, the pharmaceutical composition contains histidine at a concentration of about 50 mM.

Any suitable tonicity modifier may be used for preparing the pharmaceutical compositions described herein. In some embodiments, the tonicity modifier is a salt or an amino acid. Examples of suitable salts include, without limitation, sodium chloride, sodium succinate, sodium sulfate, potassium chloride, magnesium chloride, magnesium sulfate, and calcium chloride. In some embodiments, the tonicity modifier in the pharmaceutical composition may be at a concentration of about 10-150 mM, 50-150 mM, 50-100 mM, 75-100 mM, or about 85-95 mM. In some embodiments, the tonicity modifier may be at a concentration of about 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, or about 100 mM. In one example, the tonicity modifier may be sodium chloride, which may be at a concentration of about 90 mM.

The pharmaceutically acceptable carrier in the pharmaceutical compositions described herein may further comprise one or more pharmaceutically acceptable excipients. In general, pharmaceutically acceptable excipients are pharmacologically inactive substances. Non-limiting examples of excipients include lactose, glycerol, xylitol, sorbitol, mannitol, maltose, inositol, trehalose, glucose, bovine serum albumin (BSA), dextran, polyvinyl acetate (PVA), hydroxypropyl methylcellulose (HPMC), polyethyleneimine (PEI), gelatin, polyvinylpyrrolidone (PVP), hydroxyethylcellulose (HEC), polyethylene glycol (PEG), ethylene glycol, glycerol, dimethysulfoxide (DMSO), dimethylformamide (DMF), polyoxyethylene sorbitan monolaurate (Tween-20), polyoxyethylene sorbitan monooleate (Tween-80), sodium dodecyl sulphate (SDS), polysorbate, polyoxyethylene copolymer, potassium phosphate, sodium acetate, ammonium sulfate, magnesium sulfate, sodium sulfate, trimethylamine N-oxide, betaine, zinc ions, copper ions, calcium ions, manganese ions, magnesium ions, CHAPS, sucrose monolaurate and 2-O-beta-mannoglycerate. In some embodiments, the pharmaceutically acceptable carrier comprises an excipient between about 0.001%-0.1%, 0.001%-0.05%, 0.005-0.1%, 0.005%-0.05%, 0.008%-0.05%, 0.008%-0.03% or about 0.009%-0.02%. In some embodiments, the excipient is at about 0.005%, 0.006%, 0.007%, 0.008%, 0.009%, 0.01%, 0.02%, 0.03%, 0.04%, 0.05%, 0.06%, 0.07%, 0.08%, 0.09%, or about 0.1%. In some embodiments, the excipient is polyoxyethylene sorbitan monooleate/polysorbate 80 (Tween-80Ⓡ). In one example, the pharmaceutically acceptable carrier contains 0.01% polyoxyethylene sorbitan monooleate/polysorbate 80 (Tween-80Ⓡ).

In some examples, the pharmaceutical composition described herein comprises the anti-pKal antibody as also described herein (e.g., lanadelumab), and one or more of sodium phosphate (e.g., sodium phosphate dibasic dihydrate), citric acid (e.g., citric acid monohydrate), histidine (e.g., L-histidine), sodium chloride, and polysorbate 80. For example, the pharmaceutical composition may comprise the antibody, sodium phosphate, citric acid, histidine, sodium chloride, and polysorbate 80. In some examples, the antibody is formulated in about 30 mM sodium phosphate, about 19 mM citric acid, about 50 mM histidine, about 90 mM sodium chloride, and about 0.01% polysorbate 80. The concentration of the antibody (e.g., lanadelumab) in the composition can be about 100 mg/mL-400 mg/mL (e.g., 150 mg/mL or 300 mg/mL). In one example, the composition comprises or consists of about 150 mg lanadelumab per 1 mL solution, about 30 mM sodium phosphate dibasic dihydrate, about 19 mM (e.g., 19.6 mM) citric acid monohydrate, about 50 mM L-histidine, about 90 mM sodium chloride, and about 0.01% polysorbate 80. In another example, the composition comprises or consists of about 300 mg lanadelumab per 1 mL solution, about 30 mM sodium phosphate dibasic dihydrate, about 19 mM (e.g., 19.6 mM) citric acid monohydrate, about 50 mM L-histidine, about 90 mM sodium chloride, and about 0.01% polysorbate 80.

A pharmaceutically acceptable salt is a salt that retains the desired biological activity of the compound and does not impart any undesired toxicological effects (see, e.g., Berge, S.M., et al., 1977, J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous, and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids, and the like. Base addition salts include those derived from alkaline earth metals, such as sodium, potassium, magnesium, calcium, and the like, as well as from nontoxic organic amines, such as N,N′-dibenzylethylenediamine, N-methylglucamine, chloroprocaine, choline, diethanolamine, ethylenediamine, procaine, and the like.

The compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form can depend on the intended mode of administration and therapeutic application. Many compositions are in the form of injectable or infusible solutions, such as compositions similar to those used for administration of humans with antibodies. An exemplary mode of administration is parenteral (e.g., intravenous, subcutaneous, intraperitoneal, intramuscular). In one embodiment, the plasma kallikrein antibody is administered by intravenous infusion or injection. In another embodiment, the plasma kallikrein antibody is administered by intramuscular injection. In another embodiment, the plasma kallikrein antibody is administered by subcutaneous injection. In another preferred embodiment, the plasma kallikrein antibody is administered by intraperitoneal injection.

The phrases “parenteral administration” and “administered parenterally,” as used herein, means modes of administration other than enteral and topical administration, usually by injection, and includes, without limitation, intravenous (including intravenous infusion), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. In some embodiments, the antibody is administered subcutaneously.

The composition can be formulated as a solution, microemulsion, dispersion, liposome, or other ordered structure suitable to high drug concentration. Sterile injectable solutions can be prepared by incorporating the binding protein in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

An antibody as described herein (e.g., lanadelumab) can be administered by a variety of methods, including intravenous injection, subcutaneous injection, or infusion. The route and/or mode of administration will vary depending upon the desired results. In certain embodiments, the active compound may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are available. See, e.g., Sustained and Controlled Release Drug Delivery Systems, J.R. Robinson, ed., 1978, Marcel Dekker, Inc., New York.

Pharmaceutical compositions can be administered with medical devices. For example, in one embodiment, a pharmaceutical composition disclosed herein can be administered with a device, e.g., a needleless hypodermic injection device, a pump, or implant.

In certain embodiments, an antibody as described herein (e.g., lanadelumab) can be formulated to ensure proper distribution in vivo. For example, the blood-brain barrier (BBB) excludes many highly hydrophilic compounds. To ensure that the therapeutic compounds disclosed herein cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties that are selectively transported into specific cells or organs, thus enhance targeted drug delivery (see, e.g., V.V. Ranade, 1989, J. Clin. Pharmacol. 29:685).

Dosage regimens are adjusted to provide the optimum desired response (e.g., a therapeutic response). For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms can be dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a plasma kallikrein antibody as described herein (e.g., lanadelumab) is about 100 mg - 400 mg. In some embodiments, the therapeutically effective amount of a plasma kallikrein antibody as described herein (e.g., lanadelumab) is about 150 mg or 300 mg. In some embodiments, a therapeutically or prophylactically effective amount of an antibody is administered in a single dose. In some embodiments, a therapeutically or prophylactically effective amount of an antibody is administered in multiple doses (e.g., every two weeks for a treatment period).

As will be understood by one of ordinary skill in the art, a therapeutically or prophylactically effective amount of an antibody may be lower for a pediatric subject than for an adult subject. In some embodiments, the effective amount that is administered to a pediatric subject is a fixed dose or a weight-based dose. In some embodiments, effective amount that is less than about 150 mg or 300 mg is administered to a pediatric subject. In some embodiments, a therapeutically or prophylactically effective amount of an antibody is administered in a single dose. In some embodiments, a therapeutically or prophylactically effective amount of an antibody is administered in multiple doses (e.g., every two weeks for a treatment period).

### Kits

An antibody as described herein (e.g., lanadelumab) can be provided in a kit, e.g., as a component of a kit. For example, the kit includes (a) lanadelumab, e.g., a composition (e.g., a pharmaceutical composition) that includes the antibody, and, optionally (b) informational material. The informational material can be descriptive, instructional, marketing or other material that relates to a method described herein and/or the use of an antibody as described herein (e.g., lanadelumab), e.g., for a method described herein. In some embodiments, the kit comprises one or more doses of lanadelumab. In some embodiments, the one or more doses are 150 mg or 300 mg.

In some embodiments, the kit may further include one or more additional therapeutic agent, such as any of the additional therapeutics described herein. In some embodiments, the kit includes one or more additional inhibitors of the contact activation system (e.g., icatibant). In some embodiments, the kit includes one or more additional therapeutic agents for the treatment of acute respiratory distress syndrome. In some embodiments, the kit includes one or more additional therapeutic agents for the treatment of a viral infection (e.g., an antiviral agent).

The informational material of the kit is not limited in its form. In one embodiment, the informational material can include information about production of the compound, molecular weight of the compound, concentration, date of expiration, batch or production site information, and so forth. In one embodiment, the informational material relates to using the antibody to treat acute respiratory distress syndrome.

In one embodiment, the informational material can include instructions to administer an antibody as described herein (e.g., lanadelumab) in a suitable manner to perform the methods described herein, e.g., in a suitable dose, dosage form, mode of administration or dosing schedule (e.g., a dose, dosage form, dosing schedule or mode of administration described herein). In another embodiment, the informational material can include instructions to administer an antibody as described herein (e.g., lanadelumab) to a suitable subject, e.g., a human, e.g., a human having, or at risk for, a plasma kallikrein associated disease or condition. For example, the material can include instructions to administer an antibody as described herein (e.g., lanadelumab) to a patient with a disorder or condition described herein, e.g., a plasma kallikrein associated disease, e.g., according to a dosing schedule described herein. The informational material of the kits is not limited in its form. In many cases, the informational material, e.g., instructions, is provided in print but may also be in other formats, such as computer readable material.

An antibody as described herein (e.g., lanadelumab) can be provided in any form, e.g., liquid, dried or lyophilized form. It is preferred that an antibody be substantially pure and/or sterile. When an antibody is provided in a liquid solution, the liquid solution preferably is an aqueous solution, with a sterile aqueous solution being preferred. When an antibody is provided as a dried form, reconstitution generally is by the addition of a suitable solvent. The solvent, e.g., sterile water or buffer, can optionally be provided in the kit.

The kit can include one or more containers for the composition containing an antibody as described herein (e.g., lanadelumab). In some embodiments, the kit contains separate containers, dividers or compartments for the composition and informational material. For example, the composition can be contained in a bottle, vial, or syringe, and the informational material can be contained in association with the container. In other embodiments, the separate elements of the kit are contained within a single, undivided container. For example, the composition is contained in a bottle, vial, or syringe that has attached thereto the informational material in the form of a label. In some embodiments, the kit includes a plurality (e.g., a pack) of individual containers, each containing one or more unit dosage forms (e.g., a dosage form described herein) of an antibody as described herein (e.g., lanadelumab). For example, the kit includes a plurality of syringes, ampules, foil packets, or blister packs, each containing a single unit dose of an antibody as described herein (e.g., lanadelumab). The containers of the kits can be airtight, waterproof (e.g., impermeable to changes in moisture or evaporation), and/or light-tight.

The kit optionally includes a device suitable for administration of the composition, e.g., a syringe, or any such delivery device. In one embodiment, the device is an implantable device that dispenses metered doses of the antibody. The disclosure also features a method of providing a kit, e.g., by combining components described herein.

### EXAMPLES

### Example 1: Exploratory Study of Contact Activation System Modulation With Combination Bradykinin Receptor Blockade and Concurrent pKal Inhibition to Treat COVID-19 Pneumonia with Respiratory Distress

An open label, single arm, multi-center study is conducted to assess the safety, tolerability, and efficacy of icatibant and lanadelumab for the treatment of SARS-CoV-2 (COVID-19) pneumonia with respiratory distress in patients either at risk of or needing ventilation or recent onset of ventilatory support.

### Objective

To evaluate the safety, tolerability, and efficacy of multiple doses of icatibant, 30 milligrams (30 mg) administered subcutaneously (SC) every 8 hours (8 h) for 3 days with or without concurrent administration of lanadelumab (300 mg SC) every 2 weeks for at least one dose.

### Population

About 300 patients >18 years old with confirmed SARS-CoV-2 (COVID-19) pneumonia with respiratory distress (RD) on or off ventilator. Confirmed SARS-CoV-2 (COVID-19) diagnosis can be with any of the following:
respiratory distress, with a respiratory rate ≥ 30 breaths/min,
peripheral capillary oxygen saturation (SpO₂) ≤ 93% at rest, and/or
partial arterial oxygen pressure (PaO₂)/Fraction of inspiration O₂ (FiO₂) ≤ 300 mm Hg (1 mm Hg = 0.133 kPa).

The patients may also have chest imaging confirming lung involvement with inflammatory exudation or pleural effusion.

### Duration of Trial, Primary Endpoint, and Secondary Endpoints

The duration of the trial is 28 days with the primary endpoint being all cause mortality. The secondary endpoints include: days on ventilator or need for ventilator, in-hospital time, PaO₂/FiO₂ ratio, oxygen saturation (SaO₂), partial pressure of oxygen (PaO₂), X-ray progression, and blood pressure, specifically the need for vasopressor agents to constrict blood vessels. Safety of the treatment regimen will also be evaluated.

### Study Design #1

All patients receive open label icatibant 30 mg subcutaneously (SC) every 8 hours for 3 days and lanadelumab 300 mg SC once every 14 days.

### Study Design #2

Patients are randomly assigned into two groups: icatibant and lanadelumab (30 mg administered subcutaneously every 8 hours for 3 days and lanadelumab 300 mg administered subcutaneously once every 14 days) or icatibant only (30 mg administered subcutaneously every 8 hour for 3 days).

### Example 2: Exploratory Study of Contact Activation System Modulation with pKal Inhibition to Treat SARS-CoV-2 (COVID-19) Pneumonia with Respiratory Distress

An open label, single arm, multi-center study is conducted to assess the safety, tolerability, and efficacy of lanadelumab for the treatment of SARS-CoV-2 (COVID-19) pneumonia with respiratory distress in patients either at risk of or needing ventilation or recent onset of ventilatory support.

### Objective

To evaluate the safety, tolerability, and efficacy of lanadelumab (300 mg SC) every 2 weeks for at least one dose.

### Population

About 300 patients >18 years old with confirmed SARS-CoV-2 (COVID-19) pneumonia with respiratory distress (RD) on or off ventilator. Confirmed SARS-CoV-2 (COVID-19) diagnosis can be with any of the following:
respiratory distress, with a respiratory rate ≥ 30 breaths/min,
peripheral capillary oxygen saturation (SpO₂) ≤ 93% at rest, and/or
partial arterial oxygen pressure (PaO₂)/Fraction of inspiration O₂ (FiO₂) ≤ 300 mm Hg (1 mm Hg = 0.133 kPa).

The patients may also have chest imaging confirming lung involvement with inflammatory exudation or pleural effusion.

### Duration of Trial, Primary Endpoint, and Secondary Endpoints

All patients receive lanadelumab 300 mg SC once every 14 days. The duration of the trial is 28 days with the primary endpoint being all cause mortality. The secondary endpoints include: days on ventilator or need for ventilator, in-hospital time, PaO₂/FiO₂ ratio, oxygen saturation (SaO₂), partial pressure of oxygen (PaO₂), X-ray progression, and blood pressure, specifically the need for vasopressor agents to constrict blood vessels. Safety of the treatment regimen will also be evaluated.

### Example 3: Exploratory Study of Contact Activation System Modulation with pKal Inhibition to Treat Acute Respiratory Distress syndrome

An open label, single arm, multi-center study is conducted to assess the safety, tolerability, and efficacy of lanadelumab for the treatment of acute respiratory distress syndrome (ARDS).

### Objective

To evaluate the safety, tolerability, and efficacy of lanadelumab (300 mg SC) every 2 weeks for at least one dose.

### Population

About 300 patients >18 years old with acute respiratory distress syndrome with respiratory distress on or off ventilator. A subject with ARDS may have any of the following:
respiratory distress, with a respiratory rate ≥ 30 breaths/min,
peripheral capillary oxygen saturation (SpO₂) ≤ 93% at rest, and/or
partial arterial oxygen pressure (PaO₂)/Fraction of inspiration O₂ (FiO₂) ≤ 300 mm Hg (1 mm Hg = 0.133 kPa).

### Duration of Trial, Primary Endpoint, and Secondary Endpoints

All patients receive lanadelumab 300 mg SC once every 14 days. The duration of the trial is 28 days with the primary endpoint being all cause mortality. The secondary endpoints include: days on ventilator or need for ventilator, in-hospital time, PaO₂/FiO₂ ratio, oxygen saturation (SaO₂), partial pressure of oxygen (PaO₂), X-ray progression, and blood pressure, specifically the need for vasopressor agents to constrict blood vessels. Safety of the treatment regimen will also be evaluated.

### Example 4: Study of Lanadelumab in Patients Hospitalized with SARS-CoV-2 (COVID-19) Pneumonia

A Phase 1b, randomized, double-blind, single and repeat dose study is conducted to evaluate the safety, pharmacokinetics, and pharmacodynamics of lanadelumab when added to standard-of-care (SoC) in subjects hospitalized with COVID-19 pneumonia. The purpose of this study is to evaluate the safety, pharmacokinetic and pharmacodynamics of lanadelumab administered by intravenous (IV) infusion when added to SoC in adults hospitalized with COVID-19 pneumonia. The study is conducted in two dosing cohorts (Single-dose Cohort, and Repeat-dose Cohort). Each dosing Cohort consists of 12 subjects randomized in 3:1 ratio (9 subjects in lanadelumab : 3 participants in placebo) to 1 of 2 treatments, lanadelumab or matching placebo for a double-blind treatment period of 15 days and a safety-follow up period of approximately 2 weeks after end of treatment.

### Treatment Arms

Subjects in the experimental arm receive 300 milligram (mg) of lanadelumab intravenous (IV) infusion on Day 1 (Single-dose Cohort) or on Day 1 and Day 4 (Repeat-dose Cohort). Subjects in the placebo arm receive placebo matching IV infusion on Day 1 (Single dose Cohort) or on Day 1 and Day 4 (Repeat-dose Cohort).

### Inclusion Criteria

Subjects must be male or female adults of 18 years of age or older at the time of signing of the informed consent form (ICF). Subjects must also:
be hospitalized with evidence of COVID-19 pneumonia defined as: SARS-CoV-2 infection documented with polymerase chain reaction (PCR) of any specimen (e.g., respiratory, blood, urine, stool, or other bodily fluid); presence of respiratory distress as indicated by peripheral capillary oxygen saturation (SpO₂) lesser than or equal to (≤) 93 percent (%) on room air or respiratory rate greater than or equal to (≥) 30 breaths per minute (breaths/min);
provide written informed consent approved by the institutional review board (IRB)/independent ethics committee (IEC) before any study-specific procedures are performed; and
agree to adhere to the protocol-defined schedule of treatments, assessments, and procedures.

### Exclusion Criteria

Subjects cannot:
have a known or suspected venous thromboembolism or hypersensitivity to lanadelumab or any of its excipients;
require vasopressor support (use of fluid support is not exclusionary);
have active tuberculosis or clinical suspicion of latent tuberculosis;
have been previously diagnosed with acquired immunodeficiency syndrome (AIDS);
have been using supplemental oxygen for a medical condition prior to receiving COVID-19 diagnosis;
have used (within 3 months of screening) / or is using immunomodulators (e.g. methotrexate, azathioprine, 6-mercaptopurine, tumor necrosis factor [TNF] alpha inhibitor, Janus kinase [JAK] inhibitor, alpha-integrin);
have been dosed with an investigational drug or exposed to an investigational device within 4 weeks prior to screening;
have been exposed to plasma kallikrein inhibitors or bradykinin receptor blocker prior to screening;
be pregnant or breastfeeding;
fail to meet the laboratory and other parameters required by the study protocol;
have invasive mechanical ventilation (IMV), extracorporeal membrane oxygenation (ECMO), or evidence of severe respiratory distress such that IMV/ECMO is imminent within 12 hours of randomization;
in the opinion of the investigator, progress to death imminently and inevitably within the next 24 hours, irrespective of the provision of treatments;
have any significant condition (any surgical or medical condition) that, in the opinion of the investigator or sponsor, may compromise their safety or compliance, preclude the successful conduct of the study, or interfere with interpretation of the results (e.g. significant pre-existing illness or other major comorbidities that the investigator considers may confound the interpretation of study results); or
have any of the following laboratory abnormalities at screening:
   a) hemoglobin ≤ 8 grams per deciliter (8 g/dL),
   b) white blood cells ≤ 3000 /microliter (µL),
   c) platelets ≤ 75,000 /µL,
   d) alanine aminotransferase (ALT) or aspartate aminotransferase (AST) ≥ 3 × upper limit of normal (ULN); alkaline phosphatase (ALP) ≥ 3 x ULN; or total bilirubin greater than (>) 2 × ULN (unless the bilirubin elevation is a result of Gilbert’s syndrome), or
   e) creatinine ≥ 2 x ULN.

### Participation Requirements

A subject’s maximum duration of participation is up to 34 days, comprising a screening period of up to 48 hours, a double-blind treatment period of up to 15 days and a safety-follow up period of approximately 2 weeks after end of treatment. All participants eligible for the study are receiving standard-of-care in line with institutional practice as well as treatment as described in the study protocol.

### Experimental Outcomes

### Primary Outcome: Safety

The number of participants with treatment emergent adverse events (TEAEs) is measured. Treatment-emergent adverse events are defined as adverse events (AEs) with onset at the time of or following the start of treatment with lanadelumab, or medical conditions present prior to the start of treatment but increasing in severity or relationship at the time of or following the start of treatment. Severe adverse events (SAE) are any untoward clinical manifestation of signs, symptoms or outcomes (whether considered related to investigational product or not and at any dose: results in death, is life-threatening, requires inpatient hospitalization or prolongation of hospitalization, results in persistent or significant disability/incapacity, congenital abnormality/birth defect, an important medical event). An adverse event of special interest (AESI) includes hypersensitivity reactions, events of disordered coagulation such as bleeding AESI, hypercoagulable AESI. The number of participants with TEAEs including AESI and SAE is assessed. Safety is monitored from the start of lanadelumab administration to follow-up (up to Day 29 following lanadelumab administration).

### Secondary Outcome: Pharmacokinetics (PK) of Plasma Lanadelumab

The PK plasma concentrations of lanadelumab after a single and repeat intravenous (IV) doses is being measured. Samples are being obtained from the Single-dose Cohort and the Repeat-dose Cohort before dosing (pre-dose) and 1 hour, 24 hours, 72 hours, 144 hours, 216 hours, and 336 hours after dosing. Additional secondary outcomes include evaluating the pharmacokinetic and pharmacodynamic properties of lanadelumab and the relationship between exposure and response in subjects with COVID-19; and assessing biomarkers of the kallikrein-kinin system, cleaved high molecular weight kininogen (cHWMK), plasma kallikrein activity, prekallikrein activity, and functional C1-INH.

### Exploratory Outcomes

Further exploratory outcomes may be also be evaluated, including biomarkers associated with inflammation, such as C-reactive protein (CRP), lactate dehydrogenase (LDH), ferritin, angiopoietin-2, and cytokines, including interleukin (IL)-1β, IL-6, IL-8, IL-10, tumor necrosis factor-α (TNF-α); biomarkers associated with coagulation/fibrinolysins, including fibrinogen, D-dimer, plasminogen-activator inhibitor-1 (PAI-1), fibrin degradation products; and clinical outcomes, such as change from baseline in SpO₂/FO₂, need for mechanical ventilation, duration of hospital stay, time to hospital discharge, intensive care unit (ICU) admissions and/or duration of stay, and all-cause mortality.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of examples only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method for treating acute respiratory distress syndrome (ARDS), the method comprising:
administering to a subject in need thereof an inhibitor of the contact activation pathway.

2. The method of claim 1, wherein the subject is a human subject.

3. The method of claim 2, wherein the human subject has, or is suspected of having, a viral infection.

4. The method of claim 3, wherein the viral infection is a respiratory viral infection.

5. The method of any one of claims 1-4, wherein the ARDS is associated with a respiratory viral infection, a blood infection, pancreatitis, inhalation of toxic substances, an injury to the chest or head, or an overdose of sedatives or tricyclic antidepressants.

6. The method of claim 5, wherein the ARDS is associated with a respiratory viral infection.

7. The method of claim 6, wherein the respiratory viral infection is a coronavirus infection.

8. The method of claim 7, wherein the coronavirus infection is severe acute respiratory syndrome coronavirus 2 (SARS-CoV-2, COVID-19).

9. The method of any one of claims 1-8, wherein the subject has one or more symptom of a respiratory viral infection.

10. The method of any one of claims 1-9, wherein the subject has pneumonia associated with a respiratory viral infection.

11. The method of claim 9 or 10, wherein the one or more symptom of the respiratory viral infection is fever, cough, fatigue, sputum production, loss of smell, loss of taste, shortness of breath, muscle or joint pain, sore throat, headache, chills, nausea or vomiting, nasal congestion, diarrhea, haemoptysis, and/or conjunctival congestion.

12. The method of claim 5, wherein the ARDS is associated with inhalation of toxic substances.

13. The method of any one of claims 1-12, wherein the inhibitor of the contact activation pathway is a plasma kallikrein (pKal) inhibitor.

14. The method of claim 13, wherein the pKal inhibitor is an anti-pKal antibody.

15. The method of claim 14, wherein the anti-pKal antibody comprises heavy chain complementarity determining regions set forth by SEQ ID NOs: 5-7 and light chain variable complementarity determining regions set forth by SEQ ID NOs: 8-10.

16. The method of claim 14 or 15, wherein the anti-pKal antibody is a full-length antibody or an antigen-binding fragment thereof.

17. The method of any one of claims 14-16, wherein the anti-pKal antibody comprises a heavy chain variable region set forth by SEQ ID NO: 3 and/or a light chain variable region set forth by SEQ ID NO: 4.

18. The method of any one of claims 14-17, wherein the anti-pKal antibody comprises a heavy chain set forth by SEQ ID NO: 11 and a light chain set forth by SEQ ID NO: 12.

19. The method of any one of claims 14-18, wherein the anti-pKal antibody is formulated in a pharmaceutical composition comprising a pharmaceutical composition comprising a pharmaceutically acceptable carrier.

20. The method of claim 19, wherein the pharmaceutical composition comprises sodium phosphate, citric acid, histidine, sodium chloride, and polysorbate 80.

21. The method of claim 20, wherein the sodium phosphate is at a concentration of about 30 mM, the citric acid is at a concentration of about 19 mM, the histidine is at a concentration of about 50 mM, the sodium chloride is at a concentration of about 90 mM, and the polysorbate 80 is at about 0.01%.

22. The method of any one of claims 14-21, wherein the anti-pKal antibody is administered in one or more doses.

23. The method of claim 22, wherein each of the one or more doses comprises about 100 mg - about 400 mg of the antibody.

24. The method of claim 23, wherein each of the one or more doses comprises about 300 mg of the antibody.

25. The method of any one of claims 14-24, wherein the anti-pKal antibody is administered to the subject every two weeks.

26. The method of any one of claims 14-25, wherein the anti-pKal antibody is administered to the subject in one dose.

27. The method of any one of claims 14-26, wherein the anti-pKal antibody is administered to the subject every three days.

28. The method of any one of claims 14-27, wherein the antibody is administered subcutaneously.

29. The method of any one of claims 14-27, wherein the antibody is administered intravenously, optionally by intravenous infusion.

30. The method of any one of claims 1-29, further comprising administering one or more additional therapeutic agent to the subject.

31. The method of any one of claims 1-30, wherein the subject has been administered one or more additional therapeutic agent prior to administering the inhibitor of the contact activation pathway.

32. The method of claim 30 or 31, wherein the additional therapeutic agent is an immunomodulatory agent, an antiviral agent, an anti-malarial agent, and/or an additional inhibitor of the contact activation pathway.

33. The method of claim 32, wherein the immunomodulatory agent is an inhibitor of IL-6R.

34. The method of claim 33, wherein the inhibitor of IL-6R is tocilizumab or sarilumab.

35. The method of claim 32, wherein the antiviral agent is lopinavir, ritonavir, interferon beta, umfenovir, remdesivir, or a combination thereof.

36. The method of claim 32, wherein the anti-malarial agent is chloroquine.

37. The method of claim 32, wherein the additional inhibitor of the contact activation pathway is a Cl-inhibitor, a pKal inhibitor, or a bradykinin receptor antagonist.

38. The method of claim 37, wherein the bradykinin receptor antagonist is icatibant.

39. A method for reducing and/or preventing thrombosis associated with extracorporeal membrane oxygenation (ECMO) in a subject having acute respiratory distress syndrome (ARDS), the method comprising:
administering to the subject an inhibitor of the contact activation pathway.

40. The method of claim 39, wherein the subject is a human subject.

41. The method of claim 40, wherein the human subject has, or is suspected of having, a viral infection.

42. The method of claim 41, wherein the viral infection is a respiratory viral infection.

43. The method of any one of claims 39-42, wherein the ARDS is associated with a respiratory viral infection, a blood infection, pancreatitis, inhalation of toxic substances, an injury to the chest or head, or an overdose of sedatives or tricyclic antidepressants.

44. The method of claim 43, wherein the ARDS is associated with a respiratory viral infection.

45. The method of claim 44, wherein the respiratory viral infection is a coronavirus infection.

46. The method of claim 45, wherein the coronavirus infection is severe acute respiratory syndrome coronavirus 2 (SARS-CoV-2, COVID-19).

47. The method of any one of claims 39-46, wherein the subject has one or more symptom of a respiratory viral infection.

48. The method of any one of claims 39-47, wherein the subject has pneumonia associated with a respiratory viral infection.

49. The method of claim 47 or 48, wherein the one or more symptom of the respiratory viral infection is fever, cough, fatigue, sputum production, loss of smell, loss of taste, shortness of breath, muscle or joint pain, sore throat, headache, chills, nausea or vomiting, nasal congestion, diarrhea, haemoptysis, and/or conjunctival congestion.

50. The method of claim 43, wherein the ARDS is associated with inhalation of toxic substances.

51. The method of any one of claims 39-50, wherein the inhibitor of the contact activation pathway is a plasma kallikrein (pKal) inhibitor.

52. The method of claim 51, wherein the pKal inhibitor is an anti-pKal antibody.

53. The method of claim 52, wherein the anti-pKal antibody comprises heavy chain complementarity determining regions set forth by SEQ ID NOs: 5-7 and light chain variable complementarity determining regions set forth by SEQ ID NOs: 8-10.

54. The method of claim 52 or 53, wherein the anti-pKal antibody is a full-length antibody or an antigen-binding fragment thereof.

55. The method of any one of claims 52-54, wherein the anti-pKal antibody comprises a heavy chain variable region set forth by SEQ ID NO: 3 and/or a light chain variable region set forth by SEQ ID NO: 4.

56. The method of any one of claims 52-55, wherein the anti-pKal antibody comprises a heavy chain set forth by SEQ ID NO: 11 and a light chain set forth by SEQ ID NO: 12.

57. The method of any one of claims 52-56, wherein the anti-pKal antibody is formulated in a pharmaceutical composition comprising a pharmaceutical composition comprising a pharmaceutically acceptable carrier.

58. The method of claim 57, wherein the pharmaceutical composition comprises sodium phosphate, citric acid, histidine, sodium chloride, and polysorbate 80.

59. The method of claim 58, wherein the sodium phosphate is at a concentration of about 30 mM, the citric acid is at a concentration of about 19 mM, the histidine is at a concentration of about 50 mM, the sodium chloride is at a concentration of about 90 mM, and the polysorbate 80 is at about 0.01%.

60. The method of any one of claims 52-59, wherein the anti-pKal antibody is administered in one or more doses.

61. The method of claim 60, wherein each of the one or more doses comprises about 100 mg - about 400 mg of the antibody.

62. The method of claim 61, wherein each of the one or more doses comprises about 300 mg of the antibody.

63. The method of any one of claims 52-62, wherein the anti-pKal antibody is administered to the subject every two weeks.

64. The method of any one of claims 52-62, wherein the anti-pKal antibody is administered to the subject in one dose.

65. The method of any one of claims 52-62, wherein the anti-pKal antibody is administered to the subject every three days.

66. The method of any one of claims 52-65, wherein the anti-pKal antibody is administered subcutaneously.

67. The method of any one of claims 52-65, wherein the anti-pKal antibody is administered intravenously, optionally by intravenous infusion.

68. The method of any one of claims 39-67, further comprising administering one or more additional therapeutic agent to the subject.

69. The method of any one of claims 39-68, wherein the subject has been administered one or more additional therapeutic agent prior to administering the inhibitor of the contact activation pathway.

70. The method of claim 68 or 69, wherein the additional therapeutic agent is an immunomodulatory agent, an antiviral agent, an anti-malarial agent, and/or an additional inhibitor of the contact activation pathway.

71. The method of claim 70, wherein the immunomodulatory agent is an inhibitor of IL-6R.

72. The method of claim 71, wherein the inhibitor of IL-6R is tocilizumab or sarilumab.

73. The method of claim 70, wherein the antiviral agent is lopinavir, ritonavir, interferon beta, umfenovir, remdesivir, or a combination thereof.

74. The method of claim 70, wherein the anti-malarial agent is chloroquine.

75. The method of claim 70, wherein the additional inhibitor of the contact activation pathway is a Cl-inhibitor, a pKal inhibitor, or a bradykinin receptor antagonist.

76. The method of claim 75, wherein the bradykinin receptor antagonist is icatibant.

77. A method for treating pneumonia, the method comprising:
administering to a subject in need thereof an inhibitor of the contact activation pathway.

78. The method of claim 77, wherein the subject is a human subject.

79. The method of claim 78, wherein the human subject has, or is suspected of having, a viral infection.

80. The method of claim 79, wherein the viral infection is a respiratory viral infection.

81. The method of claim 80, wherein the respiratory viral infection is a coronavirus infection.

82. The method of claim 81, wherein the coronavirus infection is severe acute respiratory syndrome coronavirus 2 (SARS-CoV-2, COVID-19).

83. The method of any one of claims 77-82, wherein the subject has one or more symptom of a respiratory viral infection.

84. The method of claim 83, wherein the one or more symptom of the respiratory viral infection is fever, cough, fatigue, sputum production, loss of smell, loss of taste, shortness of breath, muscle or joint pain, sore throat, headache, chills, nausea or vomiting, nasal congestion, diarrhea, haemoptysis, and/or conjunctival congestion.

85. The method of any one of claims 77-84, wherein the inhibitor of the contact activation pathway is a plasma kallikrein (pKal) inhibitor.

86. The method of claim 85, wherein the pKal inhibitor is an anti-pKal antibody.

87. The method of claim 86, wherein the anti-pKal antibody comprises heavy chain complementarity determining regions set forth by SEQ ID NOs: 5-7 and light chain variable complementarity determining regions set forth by SEQ ID NOs: 8-10.

88. The method of claim 86 or 87, wherein the anti-pKal antibody is a full-length antibody or an antigen-binding fragment thereof.

89. The method of any one of claims 86-88, wherein the anti-pKal antibody comprises a heavy chain variable region set forth by SEQ ID NO: 3 and/or a light chain variable region set forth by SEQ ID NO: 4.

90. The method of any one of claims 86-89, wherein the anti-pKal antibody comprises a heavy chain set forth by SEQ ID NO: 11 and a light chain set forth by SEQ ID NO: 12.

91. The method of any one of claims 86-90, wherein the anti-pKal antibody is formulated in a pharmaceutical composition comprising a pharmaceutical composition comprising a pharmaceutically acceptable carrier.

92. The method of claim 91, wherein the pharmaceutical composition comprises sodium phosphate, citric acid, histidine, sodium chloride, and polysorbate 80.

93. The method of claim 92, wherein the sodium phosphate is at a concentration of about 30 mM, the citric acid is at a concentration of about 19 mM, the histidine is at a concentration of about 50 mM, the sodium chloride is at a concentration of about 90 mM, and the polysorbate 80 is at about 0.01%.

94. The method of any one of claims 86-93, wherein the anti-pKal antibody is administered in one or more doses.

95. The method of claim 94, wherein each of the one or more doses comprises about 100 mg - about 400 mg of the antibody.

96. The method of claim 95, wherein each of the one or more doses comprises about 300 mg of the antibody.

97. The method of any one of claims 86-96, wherein the anti-pKal antibody is administered to the subject in one dose.

98. The method of any one of claims 86-96, wherein the anti-pKal antibody is administered to the subject every three days.

99. The method of any one of claims 86-96, wherein the anti-pKal antibody is administered to the subject every two weeks.

100. The method of any one of claims 86-99, wherein the antibody is administered intravenously, optionally by intravenous infusion.

101. The method of any one of claims 77-100, further comprising administering one or more additional therapeutic agent to the subject.

102. The method of any one of claims 77-101, wherein the subject has been administered one or more additional therapeutic agent prior to administering the inhibitor of the contact activation pathway.

103. The method of claim 102, wherein the additional therapeutic agent is an antiviral agent and/or an anti-malarial agent.

104. The method of claim 102, wherein the antiviral agent is lopinavir, ritonavir, interferon beta, umfenovir, remdesivir, or a combination thereof.

105. The method of claim 103, wherein the anti-malarial agent is chloroquine.


SEQUENCE LISTING (12 sequences)

SEQ ID NO: 1 - protein, 469 aa, Homo sapiens
MGWSCILFLVATATGAHSEVQLLESGGGLVQPGGSLRLSCAASGFTFSHYIMMWVRQAPGKGLEWVSGIYSSGGITVYADSVKGRFTISRDNSKNTLYLQMNSLRAEDTAVYYCAYRRIGVPRRDEFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 231 aa, Homo sapiens
MGWSCILFLVATATGAHSDIQMTQSPSTLSASVGDRVTITCRASQSISSWLAWYQQKPGKAPKLLIYKASTLESGVPSRFSGSGSGTEFTLTISSLQPDDFATYYCQQYNTYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 122 aa, Homo sapiens
EVQLLESGGGLVQPGGSLRLSCAASGFTFSHYIMMWVRQAPGKGLEWVSGIYSSGGITVYADSVKGRFTISRDNSKNTLYLQMNSLRAEDTAVYYCAYRRIGVPRRDEFDIWGQGTMVTVSS

SEQ ID NO: 4 - protein, 112 aa, Homo sapiens
DIQMTQSPSTLSASVGDRVTITCRASQSISSWLAWYQQKPGKAPKLLIYKASTLESGVPSRFSGSGSGTEFTLTISSLQPDDFATYYCQQYNTYWTFGQGTKVEIKSEQIDN

SEQ ID NO: 5 - protein, 5 aa, Homo sapiens
HYIMM

SEQ ID NO: 6 - protein, 17 aa, Homo sapiens
GIYSSGGITVYADSVKG

SEQ ID NO: 7 - protein, 13 aa, Homo sapiens
RRIGVPRRDEFDI

SEQ ID NO: 8 - protein, 11 aa, Homo sapiens
RASQSISSWLA

SEQ ID NO: 9 - protein, 7 aa, Homo sapiens
KASTLES

SEQ ID NO: 10 - protein, 8 aa, Homo sapiens
QQYNTYWT

SEQ ID NO: 11 - protein, 451 aa, Homo sapiens
EVQLLESGGGLVQPGGSLRLSCAASGFTFSHYIMMWVRQAPGKGLEWVSGIYSSGGITVYADSVKGRFTISRDNSKNTLYLQMNSLRAEDTAVYYCAYRRIGVPRRDEFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 213 aa, Homo sapiens
DIQMTQSPSTLSASVGDRVTITCRASQSISSWLAWYQQKPGKAPKLLIYKASTLESGVPSRFSGSGSGTEFTLTISSLQPDDFATYYCQQYNTYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC